(19) United States
(12) Patent Application Publication
(10) Pub. No.: US 2011/0117552 A1
(43) Pub. Date: May 19, 2011

(54) METHODS OF USING A NOD2/CARD15 HAPLOTYPE TO DIAGNOSE CROHN'S DISEASE

(75) Inventors: Kent D. Taylor, Ventura, CA (US); Jerome I. Rotter, Los Angeles, CA (US); Huiying Yang, Cerritos, CA (US); Kazuhito Sugimura, Los Angeles, CA (US); Stephan R. Targan, Santa Monica, CA (US)

(73) Assignee: Cedars-Sinai Medical Center, Los Angeles, CA (US)

(21) Appl. No.: 12/902,111

(22) Filed: Oct. 11, 2010

Related U.S. Application Data
(63) Continuation of application No. 10/274,300, filed on Oct. 18, 2002, now abandoned.

(51) Int. Cl. C12Q 1/68

(52) U.S. Cl. 435/6

(57) ABSTRACT

The present invention provides a method of diagnosing or predicting susceptibility to Crohn's disease in an individual by determining the presence or absence in the individual of a disease-predisposing haplotype containing a JW1 variant allele at the NOD2/CARD15 locus, where the presence of the disease-predisposing haplotype is diagnostic of or predictive of susceptibility to Crohn's disease.

### CROSS-REFERENCE TO RELATED APPLICATIONS

This application is a continuation of U.S. application Ser. No. 10/274,300, filed Oct. 18, 2002, which is herein incorporated by reference for all purposes.

### STATEMENT OF FEDERALLY SPONSORED RESEARCH OR DEVELOPMENT

This work was supported by grant DK46763 and DK54967 awarded by NIDDK. The United States government has certain rights in this invention.

### BACKGROUND OF THE INVENTION

1. Field of the Invention

The invention relates generally to the fields of autoimmunity and autoimmune disease and, more specifically, to genetic methods for diagnosing Crohn's disease, psoriasis, type I diabetes and other autoimmune diseases.

2. Background Information

Inflammatory bowel disease (IBD) is the collective term used to describe two gastrointestinal disorders of unknown etiology: Crohn's disease (CD) and ulcerative colitis (UC). The course and prognosis of IBD, which occurs world-wide and is reported to afflict as many as two million people, varies widely. Onset of IBD is predominantly in young adulthood with diarrhea, abdominal pain, and fever the three most common presenting symptoms. The diarrhea may range from mild to severe, and anemia and weight loss are additional common signs of IBD. Ten percent to fifteen percent of all patients with IBD will require surgery over a ten year period. In addition, patients with IBD are at increased risk for the Development of intestinal cancer. Reports of an increasing occurrence of psychological problems, including anxiety and depression, are perhaps not surprising symptoms of what is often a debilitating disease that strikes people in the prime of life.

A battery of laboratory, radiological, and endoscopic evaluations are typically combined to derive a diagnosis of IBD and to assess the extent and severity of the disease. Nevertheless, differentiating Crohn's disease from ulcerative colitis, as well as other types of inflammatory conditions of the bowel, such as irritable bowel syndrome, infectious diarrhea, rectal bleeding, radiation colitis and the like, is difficult because the mucosa of the small and large intestines reacts in a similar way to a large number of different insults. Furthermore, the extensive and often protracted clinical testing required to diagnose Crohn's disease delays accurate diagnosis and treatment and involves invasive procedures such as endoscopy.

Although its pathogenesis has not been fully elucidated, Crohn's disease appears to be a multi-factorial disease, with both environmental and genetic factors contributing to its etiology. The role of genetic factors has been supported, for example, by twin, familial clustering, and ethnic variation studies. With regard to ethnic variation, a consistently increased incidence of Crohn's disease has been documented in the Jewish population compared with other ethnic groups in the same geographic areas. These observations are considered to be evidence for a strong genetic component to the etiology of Crohn's disease and suggest that the higher risk of Crohn's disease in the Jewish population is due, at least in part, to genetic factors.

Using genome wide scanning strategies, a region of chromosome 16 has been identified which shows significant linkage to a Crohn's disease susceptibility locus. This locus, designated IBD1, contains a gene which is known alternatively as NOD2 or CARD15 and which encodes a protein involved in activation of the immune system. Several genetic variations, or “single nucleotide polymorphisms” (SNPs), that correlate with Crohn's disease have been detected in the NOD2/CARD15 gene. In particular, variations at three principal SNPs in the coding region of NOD2/CARD15 correlate with an increased incidence of Crohn's disease. However, variations at the three major NOD2/CARD15 SNPs are not present in the majority of Crohn's disease patients. In addition, the three principal NOD2/CARD15 SNPs do not account for all of the linkage between Crohn's disease and the IBD1 locus, since residual evidence of linkage in the IBD1 region is observed after these SNPs are removed from the study set.

Identification of genetic markers that are closely associated with a predisposing mutation to an autoimmune disease such as Crohn's disease can be used to design diagnostic genetic tests. Unfortunately, the genetic markers identified to date are not useful in the majority of patients with Crohn's disease. In addition, sub-populations of Crohn's disease patients can have different predisposing mutations, making it necessary to identify new genetic markers for diagnosis of specific patient sub-populations. A reliable genetic test for Crohn's disease would be highly prized as a non-invasive method for the early diagnosis of Crohn's disease and would also be useful for predicting susceptibility to Crohn's disease in asymptomatic individuals, making prophylactic therapy possible. The present invention satisfies this need and provides related advantages as well.

### SUMMARY OF THE INVENTION

The present invention provides a method of diagnosing or predicting susceptibility to Crohn's disease in an individual by determining the presence or absence in the individual of a disease-predisposing haplotype containing a JW1 variant allele at the NOD2/CARD15 locus, where the presence of the disease-predisposing haplotype is diagnostic of or predictive of susceptibility to Crohn's disease. In a method of the invention, an individual to be diagnosed can be, for example, an Ashkenazi Jew or an individual of Middle European descent. A disease-predisposing haplotype useful in the invention can further include a 268S allele, or can include a variant allele such as a JW15, JW16, JW17 or JW18 variant allele. In further embodiments, the disease-predisposing haplotype further includes a SNP 8, SNP 12 or SNP 13 “1” allele, or includes the three SNP 8, SNP 12, and SNP 13 “1” alleles.

In one embodiment, the disease-predisposing haplotype is associated with Crohn's disease in an Ashkenazi Jewish population with an odds ratio of at least 5 and a lower 95% confidence limit greater than 1. In another embodiment, the disease-predisposing haplotype is associated with Crohn's disease in an Ashkenazi Jewish population with a population attributable risk value of at least 9.

Further provided herein is a method of diagnosing or predicting susceptibility to Crohn's disease in an individual by determining the presence or absence in the individual of a disease-predisposing haplotype containing a 268S allele and a JW1 variant allele at the NOD2/CARD15 locus, where the presence of the disease-predisposing haplotype is diagnostic of or predictive of susceptibility to Crohn's disease. An individual to be diagnosed according to a method of the invention can be, for example, an Ashkenazi Jew or can be, for example, an individual of Middle European descent. In one embodiment, the disease-predisposing haplotype further includes a JW15, JW16, JW17 or JW18 variant allele. In further embodiments, the disease-predisposing haplotype additionally includes a SNP 8, SNP 12 or SNP 13 “1” allele, or includes the three SNP 8, SNP 12, and SNP 13 “1” alleles.

In another embodiment, the disease-predisposing haplotype containing a 268S allele and a JW1 variant allele at the NOD2/CARD15 locus is associated with Crohn's disease in an Ashkenazi Jewish population with an odds ratio of at least 5 and a lower 95% confidence limit greater than 1. In a further embodiment, the disease-predisposing haplotype is associated with Crohn's disease in an Ashkenazi Jewish population with a population attributable risk value of at least 9.

The presence or absence of a disease-predisposing haplotype useful in the invention can be determined, for example, using enzymatic amplification of nucleic acid from the individual. In one embodiment, the presence or absence of the disease-predisposing haplotype is determined using polymerase chain reaction amplification. In further embodiments, the polymerase chain reaction amplification is performed using one or more fluorescently labeled probes or using one or more probes which include a DNA minor grove binder. The presence or absence of the disease-predisposing haplotype also can be determined, for example, by sequence analysis. Where the disease-predisposing haplotype contains a 268S allele and a JW1 variant, the presence or absence or the haplotype can be determined, for example, by (a) obtaining material containing nucleic acid including the NOD2/CARD15 locus from the individual; (b) determining the presence or absence of a 268S allele in the material using the polymerase chain reaction; and (c) determining the presence or absence of a JW1 variant allele in the material using DNA sequence analysis.

The present invention additionally provides a method of diagnosing or predicting susceptibility to Crohn's disease in an individual by determining the presence or absence in the individual of a JW1 variant allele at the NOD2/CARD15 locus, where the presence of the JW1 variant allele is diagnostic of or predictive of susceptibility to Crohn's disease. Such a method can be useful, for example, for diagnosing an individual who is an Ashkenazi Jew or who is of Middle European descent. In one embodiment, a method of the invention further includes determining the presence or absence in the individual of a 268S allele at the NOD2/CARD15 locus, where the presence of the JW1 variant allele and the presence of the 268S allele is diagnostic of or predictive of susceptibility to Crohn's disease.

A variety of means can be useful for determining the presence or absence of a JW1 variant allele, including, for example, enzymatic amplification of nucleic acid from the individual or sequence analysis. In one embodiment, the presence or absence of a JW1 variant allele is determined using polymerase chain reaction amplification. In another embodiment, the polymerase chain reaction amplification is performed using one or more fluorescently labeled probes. In a further embodiment, the polymerase chain reaction amplification is performed using one or more probes that include a DNA minor grove binder.

Also provided herein is a method of diagnosing or predicting susceptibility to Crohn's disease in an individual by determining the presence or absence in the individual of a disease-predisposing allele linked to a JW1 variant allele at the NOD2/CARD15 locus, provided that when the disease-predisposing allele is combined in a haplotype with a 268S allele, the haplotype is associated with Crohn's disease in an Ashkenazi Jewish population with a PAR value of at least 9, where the presence of the disease-predisposing allele is diagnostic of or predictive of susceptibility to Crohn's disease. The disease-predisposing allele can be located in a coding or non-coding region of NOD2/CARD15 and can be, for example, a JW1 variant allele or a JW15, JW16, JW17 or JW18 variant allele. In one embodiment, a disease-predisposing allele useful in the invention is located in a promoter region of NOD2/CARD15.

A method of the invention that relies on a disease-predisposing allele such as JW1, JW15, JW16, JW17 or JW18 can be useful, for example, for diagnosing or predicting susceptibility to Crohn's disease in an individual who is an Ashkenazi Jew or an individual of Middle European descent. In one embodiment, the disease-predisposing allele is associated with Crohn's disease with an odds ratio of at least 5 and a lower 95% confidence limit greater than 1. In another embodiment, the disease-predisposing allele is associated with Crohn's disease in an Ashkenazi Jewish population with a PAR value of at least 15.

Any of a variety of means can be useful for determining the presence or absence of a disease-predisposing allele in a method of the invention including, for example, enzymatic amplification of nucleic acid from the individual or sequence analysis. In one embodiment, the presence or absence of a disease-predisposing allele is determined using polymerase chain reaction amplification. The polymerase chain reaction amplification can be performed, if desired, using one or more fluorescently labeled probes or using one or more probes which include a DNA minor grove binder.

A method of the invention can optionally include determining the presence or absence in the individual of a 268S allele at the NOD2/CARD15 locus. A method of the invention also can optionally include determining the presence or absence in the individual of “1” allele at SNP 8, SNP 12, or SNP 13.

### BRIEF DESCRIPTION OF THE DRAWINGS

FIG. 1 shows the mean allele sharing (MAS) in Crohn's disease multiplex families before and after stratification based on the presence of a “2” allele at SNPs 8, 12, and 13 (SNP 8,12,13+). Six microsatellite markers spanning NOD2/CARD15 locus were genotyped (from left to right: D16S403, D16S753, D16S409, D16S411, D16S419, and D16S408, respectively). The left panel shows Jewish sib pairs and the right panel shows non-Jewish sib pairs. Black circles represent the MAS from entire sib-pairs (pre-stratification) in each ethnic group. White triangles represent the MASs in the sib-pairs who do not possess any of the principal NOD2/CARD15 SNPs, and white squares represent the MASs in the sib pairs with the principal NOD2/CARD15 SNPs after stratification.

FIG. 2 shows the NOD2/CARD15 locus with the location of variants observed in the “268S alone” haplotype determined by sequence analysis of nucleic acid from 10 Ashkenazi Jewish Crohn's disease (CD) patients and 2 normal controls (NC). Patients CD1 and CD2 are homozyote patients with the 268S allele and SNP 8,12,13− (“2111”). Patients CD3-CD7 are heterozygote patients with the 268S and SNP 8,12,13− (“2111”). Patients CD8-CD10 do not contain any of the four variant alleles (“1111”), and patients NC1 and NC2 are non-variant controls (“1111”). As described herein, “1” is the common allele, and “2” is the rare allele. IVS: Intervening sequences. Alleles are designated using the numbering system of Hugot et al., Nature 411:599-603 (2001).

FIG. 3 shows the haplotype structure of five genotyped NOD2/CARD15 variants. The letters in white squares indicate the variant rare allele (“2” allele) of each SNP. The capital letters ‘S’, ‘W’, and ‘R’ represent the amino acids ‘serine’, ‘tryptophan’, and ‘arginine’ respectively. The small letter ‘t’ represents the nucleotide variant ‘thymine’ at the JW1 variant allele location, and ‘fs’ represents the frame shift mutation at SNP 13.

FIG. 4 shows the population attributable risk (PAR) of NOD2/CARD15 haplotypes in Ashkenazi Jews and non-Jewish Caucasians. The variant names in this figure represent the different haplotype defined in Tables 3 and 5. The negative PAR values of non-risk haplotype (‘268S-JW1’ in non-Jews, ‘268S-JW1(−)’ and ‘Other’ in both ethnic groups) are not shown in this figure.

FIG. 5 shows the phylogenetic tree of five haplotypes generated using CLUSTALW. In addition, the PAR values of each haplotype in Ashkenazi Jews and non-Jewish Caucasians is shown.

FIG. 6 shows the nucleotide sequence of NOD2/CARD15 surrounding SNP 8. The top strand is labeled as SEQ ID NO:1 and the bottom strand is labeled as SEQ ID NO:2. Nucleotide sequences which can be used as primers for PCR amplification are indicated. In addition, the position of a nucleotide sequence which can be used as a probe in an allelic discrimination assay is indicated, in this figure, by a box and lower-case letters. The underlined nucleotide indicates the position of the polymorphic site.

FIG. 7 shows the nucleotide sequence of NOD2/CARD15 surrounding SNP 12. The top strand is labeled as SEQ ID NO:3 and the bottom strand is labeled as SEQ ID NO:4. Nucleotide sequences which can be used as primers for PCR amplification are indicated. In addition, the position of a nucleotide sequence which can be used as a probe in an allelic discrimination assay is indicated, in this figure, by a box. The underlined nucleotide indicates the position of the polymorphic site.

FIG. 8 shows the nucleotide sequence of NOD2/CARD15 surrounding SNP 13. The top strand is labeled as SEQ ID NO:5 and the bottom strand is labeled as SEQ ID NO:6. Nucleotide sequences which can be used as primers for PCR amplification are indicated. In addition, the position of a nucleotide sequence which can be used as a probe in an allelic discrimination assay is indicated, in this figure, by a box. The underlined nucleotide indicates the position of the polymorphic site.

FIG. 9 shows the nucleotide sequence of NOD2/CARD15 surrounding SNP 5. The top strand is labeled as SEQ ID NO:7 and the bottom strand is labeled as SEQ ID NO:8. Nucleotide sequences which can be used as primers for PCR amplification are indicated. In addition, the Position of a nucleotide sequence which can be used as a probe in an allelic discrimination assay is indicated, in this figure, by a box. The underlined nucleotide indicates the position of the polymorphic site.

FIG. 10 shows the nucleotide sequence 4 of NOD2/CARD15 surrounding the JW1 variant sequence. The top strand is labeled as SEQ ID NO:9 and the bottom strand is labeled as SEQ ID NO:10. Nucleotide sequences which can be used as primers for PCR amplification are indicated. In addition, the position of a nucleotide sequence which can be used as a probe in an allelic discrimination assay is indicated, in this figure, by a box. The underlined nucleotide indicates the position of the polymorphic site.

FIG. 11 shows the nucleotide sequence of the 5′ untranslated region of NOD2/CARD15 in 12 individuals (SEQ ID NOS:12-23) compared to the wild-type NOD2/CARD15 sequence (SEQ ID NO:11). Areas of sequence identify are shaded. The location of two polymorphic sites, JW18 and JW17 are indicated.

FIG. 12 shows the nucleotide sequence of the 3′ untranslated region of NOD2/CARD15 in 12 individuals (SEQ ID NOS:25-36 and SEQ ID NOS:78-89) compared to the wild-type NOD2/CARD15 sequence (SEQ ID NO:24 and SEQ ID NO.:77). Areas of sequence identify are shaded. The location of two polymorphic sites, JW15 and JW16 are indicated.

### DETAILED DESCRIPTION OF THE INVENTION

The present invention is directed to the exciting discovery of a disease-predisposing haplotype that is closely associated with Crohn's disease in individuals of Ashkenazi Jewish ethnicity. This disease-predisposing haplotype includes or is linked to a JW1 variant allele as described further below and can be used to diagnose or predict susceptibility to Crohn's disease.

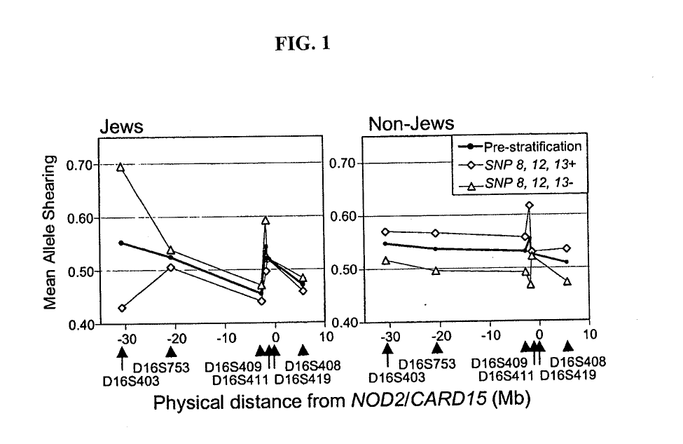

As disclosed herein, genetic linkage approaches were used to identify a strong association between a disease-predisposing haplotype and Crohn's disease in individuals of Ashkenazi Jewish ethnicity. In particular, sixty-four Ashkenazi Jewish and 147 non-Jewish Caucasian families were analyzed at six microsatellite markers spanning the IBD1 locus (see Example I and FIG. 1). These families were also genotyped for three single nucleotide polymorphisms (SNPs) in the NOD/CARD15 gene, SNP 8, SNP 12, and SNP 13, which are polymorphic markers associated with Crohn's disease in the general population. Families were divided into subgroups that contained at least one variant or “2” allele at SNP 8, 12 or 13 (SNP 8,12,13+) and subgroups that contained the wild-type or “1” allele at each of SNP 8, 12, and 13 (SNP 8,12,13−). As disclosed herein, when the Jewish families were divided into SNP 8,12,13+ and SNP 8,12,13− subgroups, a significant degree of linkage was found with microsatellite markers D16S403 and D16S411 in the SNP 8,12,13− families (see. FIG. 1). In contrast, the similarly stratified non-Jewish families did not show significant linkage at these loci. These results demonstrate that, in addition to the known SNP 8, 12, and 13 “2” alleles, one or more other IBD1 predisposing genes or NOD2/CARD15 alleles plays a more important role in susceptibility to Crohn's disease in the Jewish population as compared to the non-Jewish population.

As further disclosed herein, results obtained with an ethnically matched case-control sample showed preferential transmission of a haplotype containing the SNP 8 “1” allele, 12 “1” allele, and 13 “1” allele and a 268S allele, which is a variant allele encoding serine instead of proline at residue 268 of NOD2/CARD15. This haplotype was denoted the “268S alone” or “2111” haplotype and was associated with Crohn's disease with a p value=0.0023 (see Example II, Tables 3 and 4). In contrast, the 2111 haplotype was not associated with Crohn's disease in non-Jewish families.

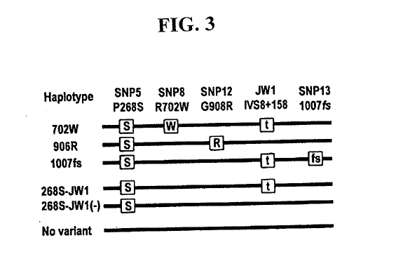

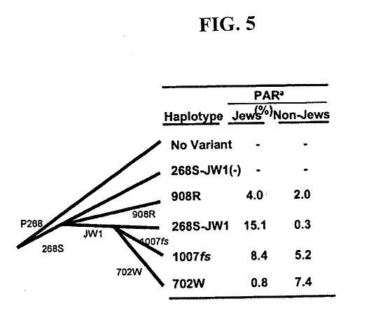

In order to identify possible unrecognized NOD2/CARD15 mutations associated with the 2111 haplotype, genomic DNA from 10 Ashkenazi Jewish Crohn's disease patients was sequenced at the NOD2/CARD15 locus. As shown in FIG. 3, a novel sequence variant designated “JW1” exhibited linkage disequilibrium with the SNP 8 “2” allele and SNP 13 “2” allele (see Examples III and IV). As further shown in Table 5, when the “JW1” variant was analyzed in conjunction with the 268S allele in the Ashkenazi Jewish population, a strong association between the 268S-JW1 haplotype and Crohn's disease was observed independent of the contribution from the “2” alleles of SNP 8, 12, and 13 (CD 20.5% vs. control 6.3%, p=0.0005). In contrast, no association between Crohn's disease and the 268S-JW1 haplotype was found in the non-Jewish population. Furthermore, as shown in FIG. 5, the 268S-JW1 haplotype group showed the highest population attributable risk (PAR, 15.1%) among all haplotypes for individuals of Ashkenazi Jewish ethnicity. Thus, the 268S-JW1 haplotype is strongly associated with Crohn's disease in Ashkenazi Jewish individuals and can be used to diagnose or predict susceptibility to Crohn's disease or related autoimmune diseases in this population.

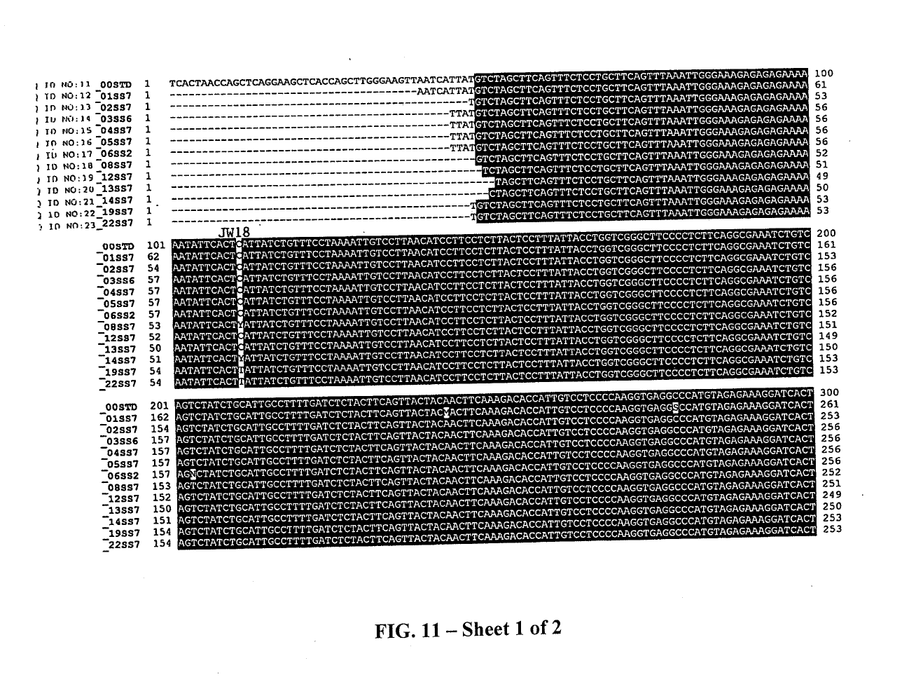

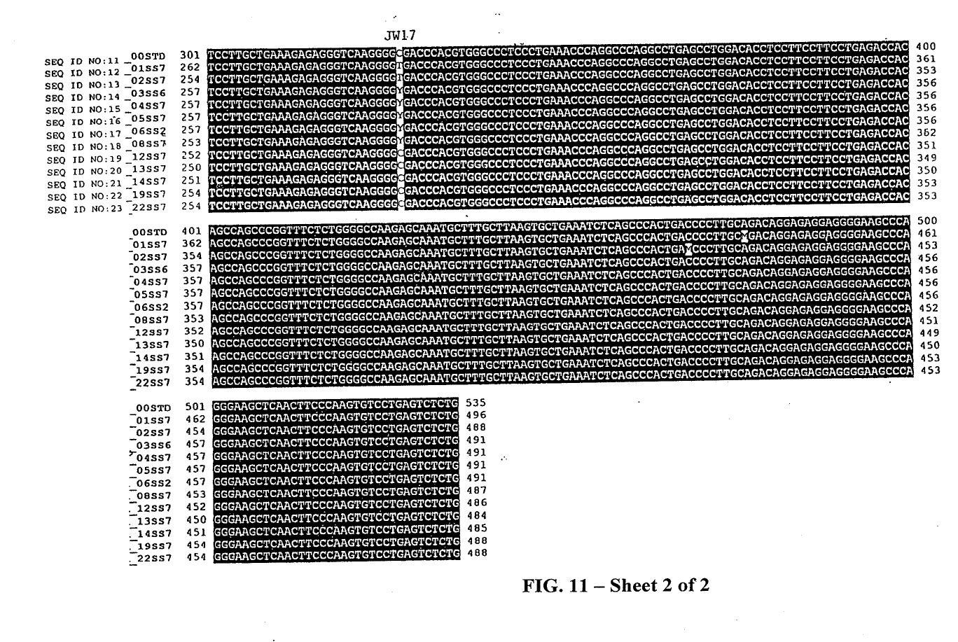

As disclosed herein, further sequence analysis of individuals with the 268S-JW1 haplotype led to the identification of additional sequence variant alleles designated JW15, JW16, JW17, and JW18. Variant alleles JW17 and JW18 are located within transcription factor binding sites in the NOD2/CARD15 5′ untranslated region, and variant alleles JW15 and JW16 are located within the NOD2/CARD15 3′ untranslated region (see FIGS. 11-13). Disease-predisposing haplotypes containing JW15, JW16, JW17, or JW18 sequence variants alleles can be associated with an autoimmune disease such as Crohn's disease either alone or in combination with other disease-predisposing alleles.

Based on these discoveries, the present invention provides a method of diagnosing or predicting susceptibility to Crohn's disease in an individual by determining the presence or absence in the individual of a JW1 variant allele at the NOD2/CARD15 locus, where the presence of the JW1 variant allele is diagnostic of or predictive of susceptibility to Crohn's disease. The invention also provides methods of diagnosing or predicting susceptibility to Crohn's disease in an individual by determining the presence or absence in the individual of a disease-predisposing haplotype containing a JW1 variant allele at the NOD2/CARD15 locus, where the presence of the disease-predisposing haplotype is diagnostic of or predictive of susceptibility to Crohn's disease. The methods of the invention are advantageous in that they are noninvasive and can be conveniently practiced, for example, with a blood sample from an individual. The methods of the invention can be used to quickly, easily and reliably diagnose or predict susceptibility to Crohn's disease, psoriasis, type I diabetes or another autoimmune diseases as disclosed herein below.

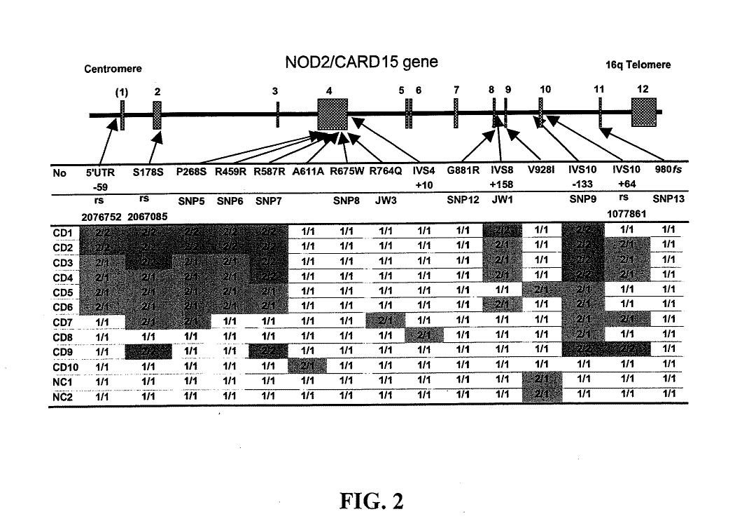

The present invention relates to genetic markers which localize to the IBD1 locus on chromosome 16. Utilizing genome wide scan linkage strategies, the IBD1 locus was mapped to the proximal region of the long arm of chromosome 16 (16q12) in the Caucasian population (Hugot et al., Nature 379:821-823 (1996)). This finding has been replicated in many studies, including an international collaborative study reporting a high multipoint linkage score (MLS) for a complex disease (MLS=5.7 at marker D16S411 in 16q12). See Cho et al., Inflamm. Bowel Dis. 3:186-190 (1997), Akolkar et al., Am. J. Gastroenterol. 96:1127-1132 (2001), Ohmen et al., Hum. Mol. Genet. 5:1679-1683 (1996), Parkes et al., Lancet 348:1588 (1996), Cavanaugh et al., Ann. Hum. Geent. (1998), Brant et al., Gastroenterology 115:1056-1061 (1998), Curran et al., Gastroenterlology 115:1066-1071 (1998), Hampe et al., Am. J. Hum. Genet. 64:808-816 (1999), and Annese et al., Eur. J. Hum. Genet. 7:567-573 (1999). The IBD1 locus has also been demonstrated to be significantly linked with Crohn's disease in Jewish families (Cho et al., supra, 1997; Akolkar et al., supra, 2001). NOD2/CARD15 within the IBD1 locus was simultaneously identified by a positional-cloning strategy (Hugot et al., Nature 411:599-603 (2001)) and a positional candidate gene strategy (Ogura et al., Nature 411:603-606 (2001), Hampe et al., Lancet 357:1925-1928 (2001)). The encoded NOD2/CARD15 protein contains amino-terminal caspase recruitment domains (CARDs) which can activate NF-κB, and several carboxy-terminal leucine-rich repeat domains (Ogura et al, J. Biol. Chem. 276:4812-4818 (2001)). FIG. 2 shows an illustration of the NOD2/CARD15 gene locus. The sequence of the human NOD2/CARD15 gene can be found in GenBank as accession number NM_{—}022162, which is incorporated by reference herein. In addition, the complete sequence of human chromosome 16 clone RP11-327F22, which includes NOD2/CARD15, can be found in GenBank as accession number AC007728, which is incorporated by reference herein. Furthermore, the sequence of NOD2/CARD15 from other species can be found in the GenBank database.

Three single nucleotide polymorphisms in the coding region of NOD2/CARD15 have been shown to be independently associated with Crohn's disease. These three SNPs, designated SNP 8, SNP 12 and SNP 13, are in the region of the NOD2/CARD15 gene which encodes the leucine-rich repeats of the NOD2/CARD15 protein (Hugot et al., supra, 2001). The rare variant or “2” alleles of the three SNP alleles are found on the same background haplotype that can be identified by several other SNPs, including 268S (previously denoted SNP 5 in Hugot et al., supra, 2001). However, an average of 60-70% of Crohn's disease patients do not have a “2” allele at SNP 8, 12, or 13. In addition, residual evidence of linkage in the IBD1 region is observed after these three SNPs are removed from the study set, indicating that variants at these three SNPs do not account for all linkage between Crohn's disease and IBD1.

As used herein, the term “SNP 8” means a single nucleotide polymorphism within exon 4 in the NOD2/CARD15 gene, which encodes amino acid 702 of the NOD2/CARD15 protein. The “1” allele, in which cytosine (c) resides at position 138,991 of the AC007728 sequence, is the common or wild-type SNP 8 allele encoding arginine at amino acid 702. The “2” allele, in which thymine (t) resides at position 138,991 of the AC007728 sequence, is a rare variant, sometimes denoted the SNP 8 “2” allele. The “2” allele at SNP 8 results in an arginine (R) to tryptophan (W) substitution at amino acid 702 of the NOD2/CARD15 protein. Accordingly, the rare “2” allele at SNP 8 is denoted “R702W” or “702W” and can also be denoted “R675W” based on the earlier numbering system of Hugot et al., supra, 2001. The NCBI SNP ID number for SNP 8 is rs2066844, which is incorporated herein by reference. As disclosed herein, the presence of allele “1” or “2” at SNP 8, or another SNP described below, can be conveniently detected, for example, by allelic discrimination assays or sequence analysis, as discussed further below.

As used herein, the term “SNP 12” means a single nucleotide polymorphism within exon 8 at in the NOD2/CARD15 gene, which encodes amino acid 908 of the NOD2/CARD15 protein. The “1” allele, in which guanine (g) resides at position 128,377 of the AC007728 sequence, is the common or wild-type SNP 12 allele encoding glycine at amino acid 908. The “2” allele, in which cytosine (c) resides at position 128,377 of the AC007728 sequence, is a rare variant, sometimes denoted as the SNP 12 “2” allele. The “2” allele at SNP 12 results in a glycine (G) to arginine (R) substitution at amino acid 908 of the NOD2/CARD15 protein. This rare “2” allele at SNP 12 is denoted “G908R” or “908R” and can also be denoted “G881R” based on the earlier numbering system of Hugot et al., supra, 2001. The NCBI SNP ID number for SNP 12 is rs2066845, which is incorporated herein by reference.

Of the three principal NOD2/CARD15 single nucleotide polymorphisms, the most significant association with Crohn's disease has been found with the “2” allele of SNP 13, which is an insertion of a single nucleotide that results in a frame shift in the tenth leucine-rich repeat of the NOD2/CARD15 protein and is followed by a premature stop codon. The resulting truncation of the NOD2/CARD15 protein appears to prevent activation of NF-κB in response to bacterial lipopolysaccharides (Ogura et al., Nature 411:603-606 (2001)). As used herein, the term “SNP 13” means a single nucleotide polymorphism within exon 11 in the NOD2/CARD15 gene, which encodes amino acid 1007 of the NOD2/CARD15 protein. The “2” allele, in which a cytosine has been added at position 121,139 of the AC007728 sequence, is a rare variant, sometimes denoted the SNP 13 “2” allele resulting in a frame shift mutation at amino acid 1007. Accordingly, the rare “2” allele at SNP 13 is denoted “1007fs” and can also be denoted “980fs” based on the earlier numbering system of Hugot et al., supra, 2001. The NCBI SNP ID number for SNP 13 is rs2066847, which is incorporated herein by reference.

As used herein, the term “268S allele” means a genetic variation of the NOD2/CARD15 gene that results in a serine at amino acid position 268 of a NOD2/CARD15 protein. The term 268S allele is used in contrast to a P268 allele, which is the wild-type or common allele at amino acid 268 in NOD2/CARD15. Because of the degeneracy of the genetic code, any of several codons such as AGC, AGT, TCA, TCG, TCC or TCT can code for serine and, thus, can encode a 268S allele. One example of a 268S allele is SNP 5, which has a single nucleotide polymorphism within exon 4 in the NOD2/CARD15 gene which encodes amino acid 268 of the NOD2/CARD15 protein. The “1” allele, in which cytosine resides at position 140,293 of the AC007728 sequence, is the common or wild-type SNP 5 allele. The “2” allele, in which thymine (t) resides at position 140,293 of the AC007728 sequence, is a rare variant, sometimes denoted the SNP 5 “2” allele, which results in a proline (P) to serine (S) substitution at amino acid 268 of the NOD2/CARD15 protein. Accordingly, the rare “2” allele at SNP 5 can be denoted “P268S” or “268S.” The NCBI SNP ID number for SNP 5 is rs2066842, which is incorporated herein by reference.

The invention relies, in part, on a newly identified polymorphism, the “JW1 variant,” within intron 8 of the NOD2/CARD15 gene. As used herein, the term “JW1 variant allele” means a genetic variation at nucleotide 158 of intervening sequence 8 (intron 8) of a NOD2/CARD15 gene. In relation to the AC007728 sequence, the JW1 variant is located at position 128,143. The genetic variation at nucleotide 158 of intron 8 can be, but is not limited to, a single nucleotide substitution, multiple nucleotide substitutions, or a deletion or insertion of one or more nucleotides. The wild type sequences has a cytosine at position 158 of intron 8; as non-limiting examples, a JW1 variant allele can have a cytosine (C) to adenine (A), cytosine to guanine (G), or cytosine to thymine (T) substitution at nucleotide 158 of intron 8. In one embodiment, the JW1 variant allele is a change from a cytosine to a thymine at nucleotide 158 of NOD2/CARD15 intron 8.

Further provided herein are newly identified variant alleles including the “JW15 variant allele,” “JW16 variant allele,” “JW17 variant allele,” and “JW18 variant allele.” As used herein, the term “JW15 variant allele” means a genetic variation in the 3′ untranslated region of NOD/CARD15 at nucleotide position 118,790 of the AC007728 sequence. The genetic variation at nucleotide 118,790 can be, but is not limited to, a single nucleotide substitution, multiple nucleotide substitutions, or a deletion or insertion of one or more nucleotides. The wild type sequence has an adenine (a) at position 118,790; as non-limiting examples, a JW15 variant allele can have an adenine (a) to cytosine (c), adenine to guanine (g), or adenine to thymine (t) substitution at nucleotide 118,790. In one embodiment, the JW15 variant allele is a change from an adenine to a cytosine at nucleotide 118,790.

As used herein, the term “JW16 variant allele” means a genetic variation in the 3′ untranslated region of NOD/CARD15 at nucleotide position 118,031 of the AC007728 sequence. The genetic variation at nucleotide 118,031 can be, but is not limited to, a single nucleotide substitution, multiple nucleotide substitutions, or a deletion or insertion of one or more nucleotides. The wild type sequence has a guanine (g) at position 118,031; as non-limiting examples, a JW16 variant allele can have a guanine (g) to cytosine (c), guanine to adenine (a), or guanine to thymine (t) substitution at nucleotide 118,031. In one embodiment, the JW16 variant allele is a change from a guanine to an adenine at nucleotide 118,031.

As used herein, the term “JW17 variant allele” means a genetic variation in the 5′ untranslated region of NOD/CARD15 at nucleotide position 154,688 of the AC007728 sequence. The genetic variation at nucleotide 154,688 can be, but is not limited to, a single nucleotide substitution, multiple nucleotide substitutions, or a deletion or insertion of one or more nucleotides. The wild type sequence has a cytosine (c) at position 154,688; as non-limiting examples, a JW17 variant allele can have a cytosine (c) to guanine (g), cytosine to adenine (a), or cytosine to thymine (t) substitution at nucleotide 154,688. In one embodiment, the JW17 variant allele is a change from a cytosine to a thymine at nucleotide 154,688.

As used herein, the term “JW18 variant allele” means a genetic variation in the 5′ untranslated region of NOD/CARD15 at nucleotide position 154,471 of the AC007728 sequence. The genetic variation at nucleotide 154,471 can be, but is not limited to, a single nucleotide substitution, multiple nucleotide substitutions, or a deletion or insertion of one or more nucleotides. The wild type sequence has a cytosine (c) at position 154,471; as non-limiting examples, a JW18 variant allele can have a cytosine (c) to guanine (g), cytosine to adenine (a), or cytosine to thymine (t) substitution at nucleotide 154,471. In one embodiment, the JW18 variant allele is a change from a cytosine to a thymine at nucleotide 154,471.

One skilled in the art recognizes that a particular polymorphic allele can be conveniently defined, for example, in comparison to a Centre d'Etude du Polymorphisme Humain (CEPH) reference individual such as the individual designated 1347-02 (Dib et al., Nature 380:152-154 (1996)), using commercially available reference DNA obtained, for example, from PE Biosystems (Foster City, Calif.). In addition, specific information on SNPs can be obtained from the dbSNP of the National Center for Biotechnology Information (NCBI).

The term “disease-predisposing haplotype,” as used herein, means a combination of alleles of closely linked loci found in a single chromosome that tends to be inherited together with Crohn's disease. In one embodiment, the disease-predisposing haplotype includes the JW1 variant allele. In another embodiment, the disease-predisposing haplotype includes the JW1 variant and the 268S allele. In a further embodiment, the disease-predisposing haplotype includes only the JW1 variant and the 268S allele. In yet a further embodiment, the disease-predisposing haplotype includes one or more additional alleles, for example, a JW15, JW16, JW17, or JW18 variant allele, together with the JW1 variant allele and the 268S allele. In still a further embodiment, the disease-predisposing haplotype is associated with Crohn's disease in the Ashkenazi Jewish population with a PAR of at least 9. In further embodiments, the disease-predisposing haplotype is associated with Crohn's disease in the Ashkenazi Jewish population with a PAR of at least 10, 11, 12, 13, 14, or 15.

As used herein, the term “individual” means an animal, such as a human or other mammal, capable of having an autoimmune disease. An individual can have one or more symptoms of an autoimmune disease or can be asymptomatic. The methods of the invention can be useful, for example, for diagnosing Crohn's disease, psoriasis, type I diabetes, or another autoimmune disease in an individual with one or more symptoms, or for predicting susceptibility to an autoimmune disease in an asymptomatic individual such as an individual at increased risk for having the an autoimmune disease.

The methods of the invention are useful for diagnosing or predicting susceptibility to an autoimmune disease such as Crohn's disease, or regional enteritis, which is a disease of chronic inflammation that can involve any part of the gastrointestinal tract. Commonly the distal portion of the small intestine (ileum) and cecum are affected. In other cases, the disease is confined to the small intestine, colon or anorectal region. Crohn's disease occasionally involves the duodenum and stomach, and more rarely the esophagus and oral cavity.

The variable clinical manifestations of Crohn's disease are, in part, a result of the varying anatomic localization of the disease. The most frequent symptoms of Crohn's disease are abdominal pain, diarrhea and recurrent fever. Crohn's disease is commonly associated with intestinal obstruction or fistula, which is an abnormal passage, for example, between diseased loops of bowel. Crohn's disease also can include extra-intestinal complications such as inflammation of the eye, joints and skin; liver disease; kidney stones or amyloidosis; and is associated with an increased risk of intestinal cancer.

Several features are characteristic of the pathology of Crohn's disease. The inflammation associated with Crohn's disease, known as transmural inflammation, involves all layers of the bowel wall. Thickening and edema, for example, typically appear throughout the bowel wall, with fibrosis also present in long-standing disease. The inflammation characteristic of Crohn's disease also is discontinuous with segments of inflamed tissue, known as “skip lesions,” separated by apparently normal intestine. Furthermore, linear ulcerations, edema, and inflammation of the intervening tissue lead to a “cobblestone” appearance of the intestinal mucosa, which is distinctive of Crohn's disease.

A hallmark of Crohn's disease is the presence of discrete aggregations of inflammatory cells, known as granulomas, which are generally found in the submucosa. About half of Crohn's disease cases display the typical discrete granulomas, while others show a diffuse granulomatous reaction or nonspecific transmural inflammation. As a result, the presence of discrete granulomas is indicative of Crohn's disease, although the absence granulomas also is consistent with the disease. Thus, transmural or discontinuous inflammation, rather than the presence of granulomas, is a preferred diagnostic indicator of Crohn's disease (Rubin and Farber, Pathology (Second Edition) Philadelphia: J.B. Lippincott Company (1994)).

As disclosed herein, the present invention provides a method of diagnosing or predicting susceptibility to Crohn's disease in an individual by determining the presence or absence in the individual of a JW1 variant allele at the NOD2/CARD15 locus, where the presence of the JW1 variant allele is diagnostic of or predictive of susceptibility to Crohn's disease. The invention also provides methods of diagnosing or predicting susceptibility to Crohn's disease in an individual by determining the presence or absence in the individual of a disease-predisposing haplotype containing a JW1 variant allele at the NOD2/CARD15 locus, where the presence of the disease-predisposing haplotype is diagnostic of or predictive of susceptibility to Crohn's disease.

A disease-predisposing haplotype containing a JW1 variant allele at the NOD2/CARD15 locus can be useful in diagnosing or predicting susceptibility to Crohn's disease in an individual. In one embodiment, a disease-predisposing haplotype containing a JW1 variant allele is used to diagnose or predict susceptibility to Crohn's disease in an individual who is an Ashkenazi Jew. Crohn's disease is significantly more common (2 to 8 fold higher) in Ashkenazi Jews than in non-Jewish Caucasians (Brant et al., Gastroenterol. 115:1056-1061 (1998)). Furthermore, among persons of Jewish ethnicity, American or European Ashkenazi Jews have a 2 to 4 fold increased risk of having this inflammatory bowel disease compared with Sephardic or Oriental Jews (Yang and Rotter in Kirschner and Shorter (Eds.), Inflammatory Bowel Disease Baltimore: Williams and Wilkins, p. 301-331 (1995); Rozen et al., Gastroenterol. 76:25-30 (1979)). The empiric risk of Crohn's disease for a first degree relative of a proband with Crohn's disease is 7.8% for Jews compared with 5.2% for non-Jews (p=0.005; Yang et al., Gut 34:517-524 (1993)). Thus, the Jewish population and especially the Ashkenazi Jewish population represents a group at increased risk for Crohn's and autoimmune diseases of related etiology.

As used herein, the term “Ashkenazi Jew” refers to an individual who is a descendant of a Jew originating from central or eastern Europe. The term Ashkenazi Jew is used in contra-distinction to a Sephardic Jew, who is a descendant of a Jew expelled from Spain, or an Oriental Jew, who is a descendant of a Babylonian Jew.

Ashkenazi Jewish identity can be established by determining the country of origin of the grandparents of an individual who describes themselves as Jewish. Jewish individuals who have, for example, three or four grandparents who originated in countries such as Austria, Bulgaria, Czechoslovakia, Germany, Hungary, Poland, Rumania, Russia and Yugoslavia, can be classified as Ashkenazi Jews. Also, for example, a family history of certain genetic diseases, such as Tay-Sachs disease, can be used to classify a Jewish individual as an Ashkenazi Jew. Methods for establishing Ashkenazi Jewish identity are well known in the art as described, for example, in Roth et al., Gastroenterol. 96:1016-1020 (1989); Roth et al., Gastroenterology 97:900-904 (1989); and Yang et al., supra, 1993.

Ashkenazi Jews can be further subdivided based on the historical geographical migration pattern of their ancestors. The Ashkenazi Jews spread into Middle Europe in the 1st to 3rd centuries with the expansion of the Roman Empire (Stroumsa, G., In Barnavi and Eliav-Feldon (eds.) A Historical Atlas of the Jewish People: From the Time of the Patriarchs to Present Schocken Books, New York, 54-55 (1992)), and then an expansion of Ashkenazi civilization occurred from Middle to Eastern Europe in the 15th-18th centuries (Bartal, In Barnavi and Eliav Feldon (eds.) A Historical Atlas of the Jewish People: From the Time of the Patriarchs to Present Schocken Books, New York, 122-123 (1992)). Based on migration patterns, for example, Ashkenazi Jews originating from Russia and Poland can be classified separately from Ashkenazi Jews originating from a Middle European country such as Austria, Bulgaria, Czechoslovakia, Germany, Hungary, Rumania or Yugoslavia.

Ashkenazi Jews of middle European origin are at increased risk of developing inflammatory bowel disease compared with Ashkenazi Jews of Polish or Russian origin (Roth et al., supra, 1989; Zlotogora et al., Gastroenterology 99:286-287 (1990)). For more than fifteen hundred years, Ashkenazi Jews lived and expanded in Middle Europe in increasingly urbanized communities. Accordingly, Crohn's disease patients in Middle Europe, such as Crohn's disease patients in Germany, commonly possess the same population-specific genetic factors as the Ashkenazi Jewish population which originated in Middle Europe. In view of the above, one skilled in the art understands that a JW1 variant, or disease-predisposing haplotype or disease-predisposing allele can be useful in diagnosing or predicting susceptibility to Crohn's or other autoimmune diseases in individuals of Middle European descent.

The term “of Middle European descent” means an individual who is a descendant of an individual who was born in a Middle European country in the 11^{th} through 20^{th} centuries. An individual of Middle European descent has on average at least one-quarter (25%) of the genetic material of an ancestor who was born in a Middle European country. As described above, Middle European countries include countries such as Austria, Bulgaria, Czechoslovakia, Germany, Hungary, Rumania and Yugoslavia. In one embodiment, an individual of Middle European descent is a descendant of an individual who was born in a Middle European country in the 15^{th} through 18^{th} centuries.

An individual of Middle European descent can have, for example, at least 25%, 50%, 75% or 100% of the genetic material of an ancestor who originated from a Middle European country. It is understood that a child has, on average, one-half (50%) of the genetic material of a parent. Likewise, a grandchild has, on average, one-fourth (25%) of the genetic material of the parent. In an example where both parents are from a Middle European country, the children would be 100% of Middle European descent. When one parent is from a Middle European country and the other parent is not, the children would be 50% of Middle European descent. In an example where one parent is from a Middle European country and the other parent is 50% of Middle European descent, the children would be 75% of Middle European descent.

As described above and disclosed herein, an increased frequency of a haplotype carrying the 268S allele and the “1” allele at SNPs 8, 12, and 13 was found in Jewish Crohn's disease patients (OR=3.13, p=0.0023, see Example II and Table 4). Therefore this haplotype, which is designated the “268S alone” or “2111” haplotype, can be used to diagnose or predict susceptibility to Crohn's or another autoimmune disease in an Ashkenazi Jew. Thus, the present invention provides a method of diagnosing or predicting susceptibility to Crohn's disease in an Ashkenazi Jew by (a) determining the presence or absence in the Ashkenazi Jew of a 268S allele at the NOD2/CARD15 locus, and (b) determining the presence or absence in the individual of a SNP 8 “1” allele, SNP 12 “1” allele, and SNP 13 “1” allele at the NOD2/CARD15 locus, where the presence of the 268S allele and presence of the SNP 8 “1” allele, SNP 12 “1” allele, and SNP 13 “1” alleles is diagnostic of or predictive of susceptibility to Crohn's disease. In other embodiments, such a method is used to diagnose or predict susceptibility to psoriasis, type I diabetes or another autoimmune disease as discussed below.

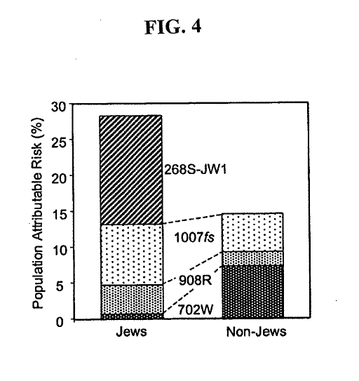

As disclosed herein, the “268S alone” haplotype can further include a JW1 variant allele, resulting in a haplotype with an increased association with Crohn's disease. In the Jewish population, a haplotype containing the JW1 variant allele and a 268S allele, designated as a 268S-JW1 haplotype or 268S+JW1 haplotype, exhibits a significant odds ratio with Crohn's disease (OR=5.75, p=0.0005) and the highest population attributable risk (PAR=15.1%) for Crohn's disease among reported alleles (see Example V and FIGS. 4 and 5). Therefore, the invention also provides a method of diagnosing or predicting susceptibility to Crohn's disease in an individual by determining the presence or absence in the individual of a JW1 variant allele and a 268S allele at the NOD2/CARD15 locus, where the presence of the JW1 variant allele together with the presence of the 268S allele is diagnostic of or predictive of susceptibility to Crohn's disease. Similarly, the invention provides a method of diagnosing or predicting susceptibility to Crohn's disease in an individual by determining the presence or absence in the individual of a disease-predisposing haplotype that includes a JW1 variant allele and a 268S allele at the NOD2/CARD15 locus, where the presence of the disease-predisposing haplotype is diagnostic of or predictive of susceptibility to Crohn's disease. As discussed further below, a disease-predisposing haplotype that includes a JW1 variant allele and a 268S allele at the NOD2/CARD15 locus also can be useful for diagnosing or predicting susceptibility to other autoimmune disease including, without limitation, psoriasis and type I diabetes.

These methods can further include determining the presence or absence of the “1” allele at a single nucleotide polymorphic site such as SNP 8, SNP 12, or SNP 13. In one embodiment, a disease-predisposing haplotype containing a JW1 variant allele and a 268S allele at a NOD2/CARD15 locus further includes a “1” allele at one or more SNPs such as SNP 8, SNP 12 and SNP 13. In another embodiment, a disease-predisposing haplotype containing a JW1 variant allele and a 268S allele at a NOD2/CARD15 locus further includes a″1″ allele at each of SNP 8, SNP 12 and SNP 13.

The invention also provides a method of diagnosing or predicting susceptibility to Crohn's disease in an individual by determining the presence or absence in the individual of a disease-predisposing haplotype containing a JW15, JW16, JW17, or JW18 variant allele at the NOD2/CARD15 locus, where the presence of the disease-predisposing haplotype is diagnostic or predictive of susceptibility to Crohn's disease. A disease-predisposing haplotype useful in the invention can contain any one or combination of the JW15, JW16, JW17, or JW18 variant alleles, alone or in combination with one or more additional alleles such as, for example, any combination of the JW1 variant allele, 268S allele, or SNP8, SNP12 or SNP13 “1” alleles.

The strength of the association between a disease-predisposing haplotype or disease-predisposing allele and Crohn's disease or another autoimmune disease can be characterized by a particular odds ratio such as an odds ratio of at least 5 with a lower 95% confidence interval limit of greater than 1. Such an odds ratio can be, for example, at least 5.5, 5.75, 6.0, 6.5, 7.0, 7.5, or 8.0 with a lower 95% confidence interval limit of greater than 1, such as an odds ratio of at least 7.5 with a 95% confidence interval of 1.6-35.5 (see Table 4). In addition, an odds ratio can be, for example, at least 3.0, at least 3.5, at least 4.0 or at least 4.5 with a lower confidence interval limit of greater than 1. Methods for determining an odds ratio are well known in the art (see, for example, Schlesselman et al., Case Control Studies Design, Conduct and Analysis Oxford University Press, New York (1982)).

In further embodiments, a disease-predisposing haplotype or disease-predisposing allele is associated with Crohn's disease or another autoimmune disease in a population such as an Ashkenazi Jewish population with a population attributable risk (PAR) value of at least 9. Within a population, a disease-predisposing haplotype or disease-predisposing allele can be associated with an autoimmune disease such as Crohn's disease in an Ashkenazi Jewish population with, for example, a PAR value of at least 9, 10, 11, 12, 13, 14, 15, 16, 20, 25, 30, 35, 40, 45, 50, or greater. Population attributable risk can be estimated assuming that the frequency of a risk haplotype in the control group can be regarded as approximately representative of the target population and the odds ratio as an approximation to the relative risk. The population attributable risk can be calculated as PAR=Pe(OR−1)/[Pe(OR−1)+1], where Pe=frequency of a risk haplotype in control group and OR=odds ratio (Schlesselman, supra, 1982).

In still further embodiments, a disease-predisposing haplotype or disease-predisposing allele is associated with Crohn's disease with a p value of equal to or less than 0.0023. As used herein, the term “p value” is synonymous with “probability value.” As is well known in the art, the expected p value for the association between a random haplotype (or allele) and disease is 1.00. A p value of less than 0.05 indicates that haplotype and disease do not appear together by chance but are influenced by positive factors. The statistical threshold for significance of linkage has been set at a level of allele sharing for which false positives would occur once in twenty genome scans (p=0.05). In particular embodiments, disease-predisposing haplotype or disease-predisposing allele is associated with an autoimmune disease such as Crohn's disease with, for example, a p value of less than 0.04, 0.03, 0.02, 0.01, 0.009, 0.008, 0.007, 0.006, 0.005, 0.004, 0.003, 0.002 or 0.001, or with a p value of less than 0.00095, 0.0009, 0.00085, 0.0008 or 0.0005. It is recognized that, in some cases, p values may need to be corrected, for example, to account for factors such as sample size (number of families), genetic heterogeneity, clinical heterogeneity, or analytical approach (parametric or nonparametric method).

A variety of means can be used to determine the presence or absence of a JW1 variant allele, or a disease-associated allele or disease-associated haplotype in a method of the invention. As an example, enzymatic amplification of nucleic acid from an individual can be conveniently used to obtain nucleic acid for subsequent analysis. The presence or absence of a JW1 variant allele, or a disease-associated allele or disease-associated haplotype also can be determined directly from the individual's nucleic acid without enzymatic amplification.

Analysis of the nucleic acid from an individual, whether amplified or not, can be performed using any of various techniques. Useful techniques include, without limitation, polymerase chain reaction based analysis, sequence analysis and electrophoretic analysis. As used herein, the term “nucleic acid” means a polynucleotide such as a single- or double-stranded DNA or RNA molecule including, for example, genomic DNA, cDNA and mRNA. The term nucleic acid encompasses nucleic acid molecules of both natural and synthetic origin as well as molecules of linear, circular or branched configuration representing either the sense or antisense strand, or both, of a native nucleic acid molecule. It is understood that such nucleic acid molecules can be attached to a synthetic material such as a bead or column matrix.

The presence or absence of a JW1 variant allele or a disease-predisposing haplotype or disease-predisposing allele can involve amplification of an individual's nucleic acid by the polymerase chain reaction. Use of the polymerase chain reaction for the amplification of nucleic acids is well known in the art (see, for example, Mullis et al. (Eds.), The Polymerase Chain Reaction, Birkhäuser, Boston, (1994)). In one embodiment, the polymerase chain reaction amplification is performed using one or more fluorescently labeled primers or using one or more labeled or unlabeled primers that contain a DNA minor grove binder.

A Taqman® allelic discrimination assay available from Applied Biosystems can be useful for determining the presence or absence of a JW1, JW15, JW16, JW17 or JW18 variant allele or another allele such as a disease-predisposing allele or an allele that is part of a disease-predisposing haplotype such as a 268S allele. In a Taqman® allelic discrimination assay, a specific, fluorescent, dye-labeled probe for each allele is constructed. The probes contain different fluorescent reporter dyes such as FAM and VIC™ to differentiate the amplification of each allele. In addition, each probe has a quencher dye at one end which quenches fluorescence by fluorescence resonant energy transfer (FRET). During PCR, each probe anneals specifically to complementary sequences in the nucleic acid from the individual. The 5′ nuclease activity of Taq polymerase is used to cleave only probe that hybridize to the allele. Cleavage separates the reporter dye from the quencher dye, resulting in increased fluorescence by the reporter dye. Thus, the fluorescence signal generated by PCR amplification indicates which alleles are present in the sample. Mismatches between a probe and allele reduce the efficiency of both probe hybridization and cleavage by Taq polymerase, resulting in little to no fluorescent signal. Improved specificity in allelic discrimination assays can be achieved by conjugating a DNA minor grove binder (MGB) group to a DNA probe as described, for example, in Kutyavin et al., “3′-minor groove binder-DNA probes increase sequence specificity at PCR extension temperature,” Nucleic Acids Research 28:655-661 (2000)). Minor grove binders include, but are not limited to, compounds such as dihydrocyclopyrroloindole tripeptide (DPI₃).

Sequence analysis also can be useful for determining the presence or absence of a JW1, JW15, JW16, JW17 or JW18 variant allele or a disease-predisposing haplotype or disease-predisposing allele in a method of the invention. The JW1 variant allele can be detected by sequence analysis using primers disclosed herein, for example, in Table 4 The term “sequence analysis,” as used herein in reference to one or more nucleic acids, means any manual or automated process by which the order of nucleotides in the nucleic acid is determined. As an example, sequence analysis can be used to determine the nucleotide sequence of a sample of DNA. The term sequence analysis encompasses, without limitation, chemical (Maxam-Gilbert) and dideoxy enzymatic (Sanger) sequencing as well as variations thereof. The term sequence analysis further encompasses, but is not limited to, capillary array DNA sequencing, which relies on capillary electrophoresis and laser-induced fluorescence detection and can be performed using, for example, the MegaBACE 1000 or ABI 3700. As additional non-limiting examples, the term sequence analysis encompasses thermal cycle sequencing (Sears et al., Biotechniques 13:626-633 (1992)); solid-phase sequencing (Zimmerman et al., Methods Mol. Cell Biol. 3:39-42 (1992); and sequencing with mass spectrometry such as matrix-assisted laser desorption/ionization time-of-flight mass spectrometry MALDI-TOF MS (Fu et al., Nature Biotech. 16: 381-384 (1998)). The term sequence analysis also includes, yet is not limited to, sequencing by hybridization (SBH), which relies on an array of all possible short oligonucleotides to identify a segment of sequences present in an unknown DNA (Chee et al., Science 274:610-614 (1996); Drmanac et al., Science 260:1649-1652 (1993); and Drmanac et al., Nature Biotech. 16:54-58 (1998)).

One skilled in the art understands that these and additional variations are encompassed by the term sequence analysis as defined herein. See, in general, Ausubel et al., supra, Chapter 7 and supplement 47.

The invention also provides a method of diagnosing or predicting susceptibility to Crohn's disease in an individual by determining the presence or absence in the individual of a disease-predisposing haplotype containing a 268S allele and a JW1 variant allele at the NOD2/CARD15 locus, where the method includes the steps of obtaining material containing nucleic acid including the NOD2/CARD15 locus from the individual; determining the presence or absence of a 268S allele in the material using the polymerase chain reaction; and determining the presence or absence of a JW1 variant allele in the material using DNA sequence analysis. As used herein, the term “material” means any biological matter from which nucleic acid molecules can be prepared. As non-limiting examples, the term material encompasses whole blood, plasma, saliva, cheek swab, or other bodily fluid or tissue that contains nucleic acid. In one embodiment, a method of the invention is practiced with whole blood, which can be obtained readily by non-invasive means and used to prepare genomic DNA, for example, for enzymatic amplification or automated sequencing. In another embodiment, a method of the invention is practiced with tissue obtained from an individual such as tissue obtained during surgery or biopsy procedures.

Electrophoretic analysis also can be useful in the methods of the invention. Elecrophoretic analysis, as used herein in reference to one or more nucleic acids such as amplified fragments, means a process whereby charged molecules are moved through a stationary medium under the influence of an electric field. Electrophoretic migration separates nucleic acids primarily on the basis of their charge, which is in proportion to their size, with smaller molecules migrating more quickly. The term electrophoretic analysis includes analysis using both slab gel electrophoresis, such as agarose or polyacrylamide gel electrophoresis, and capillary electrophoresis. Capillary electrophoretic analysis generally occurs inside a small-diameter (50-100-μm) quartz capillary in the presence of high (kilovolt-level) separating voltages with separation times of a few minutes. Using capillary electrophoretic analysis, nucleic acids are conveniently detected by UV absorption or fluorescent labeling, and single-base resolution can be obtained on fragments up to several hundred base pairs. Such methods of electrophoretic analysis, and variations thereof, are well known in the art, as described, for example, in Ausubel et al., Current Protocols in Molecular Biology Chapter 2 (Supplement 45) John Wiley & Sons, Inc. New York (1999)).

Restriction fragment length polymorphism (RFLP) analysis also can be useful for determining the presence or absence of a particular allele (Jarcho et al. in Dracopoli et al., Current Protocols in Human Genetics pages 2.7.1-2.7.5, John Wiley & Sons, New York; Innis et al., (Ed.), PCR Protocols, San Diego: Academic Press, Inc. (1990)). As used herein, restriction fragment length polymorphism analysis is any method for distinguishing genetic polymorphisms using a restriction enzyme, which is an endonuclease that catalyzes the degradation of nucleic acid and recognizes a specific base sequence, generally a palindrome or inverted repeat. One skilled in the art understands that the use of RFLP analysis depends upon an enzyme that can differentiate two alleles at a polymorphic site.

Allele-specific oligonucleotide hybridization also can be used to detect a disease-predisposing allele. Allele-specific oligonucleotide hybridization is based on the use of a labeled oligonucleotide probe having a sequence perfectly complementary, for example, to the sequence encompassing a disease-predisposing allele. Under appropriate conditions, the allele-specific probe hybridizes to a nucleic acid containing the disease-predisposing allele but does not hybridize to the one or more other alleles, which have one or more nucleotide mismatches as compared to the probe. If desired, a second allele-specific oligonucleotide probe that matches an alternate allele also can be used. Similarly, the technique of allele-specific oligonucleotide amplification can be used to selectively amplify, for example, a disease-predisposing allele by using an allele-specific oligonucleotide primer that is perfectly complementary to the nucleotide sequence of the disease-predisposing allele but which has one or more mismatches as compared to other alleles (Mullis et al., supra, (1994)). One skilled in the art understands that the one or more nucleotide mismatches that distinguish between the disease-predisposing allele and one or more other alleles are preferably located in the center of an allele-specific oligonucleotide primer to be used in allele-specific oligonucleotide hybridization. In contrast, an allele-specific oligonucleotide primer to be used in PCR amplification preferably contains the one or more nucleotide mismatches that distinguish between the disease-associated and other alleles at the 3′ end of the primer.

A heteroduplex mobility assay (HMA) is another well known assay that can be used to detect a JW1 variant allele or to detect a disease-predisposing allele or disease-predisposing haplotype in a method of the invention. HMA is useful for detecting the presence of a polymorphic sequence since a DNA duplex carrying a mismatch has reduced mobility in a polyacrylamide gel compared to the mobility of a perfectly base-paired duplex (Delwart et al., Science 262:1257-1261 (1993); White et al., Genomics 12:301-306 (1992)).

The technique of single strand conformational polymorphism (SSCP) also can be used to detect the presence or absence of a JW1 variant allele, or to detect the presence or absence of a disease-predisposing allele or disease-predisposing haplotype (see Hayashi, K., Methods Applic. 1:34-38 (1991)). This technique can be used to detect mutations based on differences in the secondary structure of single-strand DNA that produce an altered electrophoretic mobility upon non-denaturing gel electrophoresis. Polymorphic fragments are detected by comparison of the electrophoretic pattern of the test fragment to corresponding standard fragments containing known alleles.

Denaturing gradient gel electrophoresis (DGGE) also can be used to detect a JW1 variant or a disease-predisposing allele or disease-predisposing haplotype in a method of the invention. In DGGE, double-stranded DNA is electrophoresed in a gel containing an increasing concentration of denaturant; double-stranded fragments made up of mismatched alleles have segments that melt more rapidly, causing such fragments to migrate differently as compared to perfectly complementary sequences (Sheffield et al., “Identifying DNA Polymorphisms by Denaturing Gradient Gel Electrophoresis” in Innis et al., supra, 1990).

Other molecular methods useful for determining the presence or absence of a disease-predisposing allele are known in the art and useful in the methods of the invention. Other well-known approaches for determining the presence or absence of a JW1 variant allele or a disease-predisposing allele or disease-predisposing haplotype include automated sequencing and RNAase mismatch techniques (Winter et al., Proc. Natl. Acad. Sci. 82:7575-7579 (1985)). Furthermore, one skilled in the art understands that, where the presence or absence of multiple alleles or a disease-predisposing haplotype is to be determined, individual alleles can be detected by any combination of molecular methods. See, in general, Birren et al. (Eds.) Genome Analysis: A Laboratory Manual Volume 1 (Analyzing DNA) New York, Cold Spring Harbor Laboratory Press (1997). In addition, one skilled in the art understands that multiple alleles can be detected in individual reactions or in a single reaction (a “multiplex” assay). In view of the above, one skilled in the art realizes that the methods of the invention for diagnosing or predicting susceptibility to an autoimmune disease such as Crohn's disease in an individual can be practiced using one or any combination of the well known assays described above or another art-recognized genetic assay.

The invention provides a method of diagnosing or predicting susceptibility to Crohn's disease in an individual by determining the presence or absence in the individual of a disease-predisposing allele linked to a JW1 variant allele at the NOD2/CARD15 locus, provided that when the disease-predisposing allele is combined in a haplotype with a 268S allele, the haplotype is associated with Crohn's disease in an Ashkenazi Jewish population with a PAR value of at least 9, and where the presence of the disease-predisposing allele is diagnostic of or predictive of susceptibility to Crohn's disease.

The term “disease-predisposing allele,” as used herein, means a molecular variation that is linked to a JW1 variant allele and that tends to be inherited together with a disease such as Crohn's disease, and which, when combined together with a 268S allele forms a haplotype that is associated with Crohn's disease in an Ashkenazi Jewish population with a PAR value of at least 9. A disease-predisposing allele useful in the invention can be, without limitation, a single nucleotide polymorphism, a microsatellite (ms), a variable number tandem repeat (VNTR) polymorphism, or a nucleotide substitution, insertion or deletion of one or more nucleotides. One skilled in the art further understands that a disease-predisposing allele also can be a molecular variation such as abnormal methylation or other modification that does not produce a difference in the primary nucleotide sequence of the disease-predisposing allele as compared to another allele.

The term “linked to,” as used herein in reference to a disease-predisposing allele and a JW1 variant allele, means that the disease-predisposing allele and the JW1 variant allele are inherited together more often than would be expected according to traditional Mendelian genetics. The site of the disease-predisposing allele and the JW1 variant allele are in close physical proximity to each other. It is understood that two alleles are linked when there is less than 50% recombination between the two alleles. It is further understood that, since 1% recombination is roughly equivalent to 1 centiMorgan, linkage between two alleles can occur if the alleles are separated by about 50 cM or less. As an example, a disease-predisposing allele and the JW1 variant allele can be within 1 centiMorgan (cM) of each other, within 5 cM of each other, within 10 cM of each other, within 20 cM of each other, within 30 cM of each other, within 40 cM of each other, or within 50 cM of each other.

The presence of a disease-predisposing allele can be used as a surrogate for a JW1 variant allele to diagnose or predict susceptibility to an autoimmune disease such as Crohn's. For example, a disease-predisposing allele linked to a JW1 variant allele can be used as a surrogate for the JW1 variant allele to diagnose or predict susceptibility to Crohn's disease, psoriasis; type I diabetes mellitus or another autoimmune disease as described below.

Disease-predisposing alleles linked to a JW1 variant allele can be located in a coding region or non-coding region and further can be located, without limitation, in a non-coding region of the NOD2/CARD15 locus such as in a 5′ or 3′ untranslated region, an intronic sequence or in a promoter region within the 5′ untranslated region of NOD2/CARD15. In one embodiment, the disease-predisposing allele is the JW1 variant allele itself. In other embodiments, the disease-predisposing allele is a JW15, JW16, JW17, or JW18 variant allele.

In one embodiment, the invention provides a method of diagnosing or predicting susceptibility to Crohn's disease in an individual who is an Ashkenazi Jew or an individual of Middle European descent by determining the presence or absence in the individual of a disease-predisposing allele linked to a JW1 variant allele at the NOD2/CARD15 locus, provided that when the disease-predisposing allele is combined in a haplotype with a 268S allele, the haplotype is associated with Crohn's disease in an Ashkenazi Jewish population with a PAR value of at least 9, where the presence of the disease-predisposing allele is diagnostic of or predictive of susceptibility to Crohn's disease. In a further embodiment, the disease-predisposing allele is associated with Crohn's disease with an odds ratio of at least 5 and a lower 95% confidence limit greater than 1.

The invention also provides a method of diagnosing or predicting susceptibility to Crohn's disease in an individual by determining the presence or absence in the individual of a disease-predisposing allele linked to a JW1 variant allele at the NOD2/CARD15 locus and a 268S allele, provided that the disease-predisposing allele is not a SNP 8 “2” allele, SNP 12 “2” allele, or SNP 13 “2” allele, where the presence of the disease-predisposing allele is diagnostic of or predictive of susceptibility to Crohn's disease.

The methods described herein that utilize the presence of a JW1 variant allele in order to diagnose or predict susceptibility to Crohn's or another autoimmune disease can also be practiced using the R791Q variant allele that was identified herein as a novel sequence variant in an Ashkenazi Jewish Crohn's disease patient (see FIG. 2A).

The methods of the present invention are useful for diagnosing or predicting susceptibility to a variety of autoimmune diseases including, without limitation, Crohn's disease, psoriasis and Type I diabetes. As used herein, the term “autoimmune disease” means a disease resulting from an immune response against a self tissue or tissue component, and includes both self antibody responses and cell-mediated responses. The term autoimmune disease encompasses organ-specific autoimmune diseases, in which an autoimmune response is directed against a single tissue, such as Crohn's disease, psoriasis, Type I diabetes mellitus, ulcerative colitis, myasthenia gravis, vitiligo, Graves' disease, Hashimoto's disease, Addison's disease and autoimmune gastritis and autoimmune hepatitis. The term autoimmune disease also encompasses non-organ specific autoimmune diseases, in which an autoimmune response is directed against a component present in several or many organs throughout the body. Such autoimmune diseases include, without limitation, rheumatoid disease, systemic lupus erythematosus, progressive systemic sclerosis and variants, polymyositis and dermatomyositis. Additional autoimmune diseases include pernicious anemia, autoimmune gastritis, primary biliary cirrhosis, autoimmune thrombocytopenia, Sjögren's syndrome, and multiple sclerosis. One skilled in the art understands that any of the above methods of the invention can be applied to these or other organ-specific and non-organ specific autoimmune diseases.

Thus, the invention provides a method of diagnosing or predicting susceptibility to an autoimmune disease in an individual by determining the presence or absence in the individual of a JW1 variant allele at the NOD2/CARD15 locus, where the presence of the JW1 variant allele is diagnostic of or predictive of susceptibility to an autoimmune disease. The invention also provides a method of diagnosing or predicting susceptibility to an autoimmune disease in an individual by determining the presence or absence in the individual of a disease-predisposing haplotype containing a JW1 variant allele at the NOD2/CARD15 locus, where the presence of the disease-predisposing haplotype is diagnostic of or predictive of susceptibility to an autoimmune disease. In addition, the invention also provides a method of diagnosing or predicting susceptibility to an autoimmune disease in an individual by determining the presence or absence in the individual of a disease-predisposing haplotype containing a 268S allele and a JW1 variant allele at the NOD2/CARD15 locus, where the presence of the disease-predisposing haplotype is diagnostic of or predictive of susceptibility to an autoimmune disease. The invention further provides a method of diagnosing or predicting susceptibility to an autoimmune disease in an individual by determining the presence or absence in the individual of a disease-predisposing allele linked to a JW1 variant allele at the NOD2/CARD15 locus, provided that the disease-predisposing allele is not a SNP 8 “2” allele, SNP 12 “2” allele, or SNP 13 “2” allele, and where the presence of the disease-predisposing allele is diagnostic of or predictive of susceptibility to an autoimmune disease. Any of such methods of the invention for diagnosing or predicting susceptibility to an autoimmune disease are useful, for example, for diagnosis of Ashkenazi Jews or in individuals of Middle European descent. In particular embodiments, the methods of the invention are useful for diagnosing or predicting susceptibility to psoriasis or type I diabetes.

The methods of the present invention are useful for diagnosing or predicting susceptibility to psoriasis in an individual such as, without limitation, an Ashkenazi Jew or an individual or Middle European descent. Psoriasis is a chronic inflammatory skin disease most often characterized by patches of red, raised skin which can be covered with flaky dry skin called scale. The term psoriasis encompasses the most common form, plaque psoriasis, as well as other forms such as guttate psoriasis, inverse psoriasis, erythrodermic psoriasis, and generalized or localized pustular psoriasis. The term psoriasis also encompasses disease of any severity, including mild psoriasis, which is defined as psoriasis on less than 2% of the body surface; moderate psoriasis, which affects 2-10% of the body surface; and severe psoriasis, which can affect more than 10% of the body surface.

The methods of the present invention are also useful for diagnosing or predicting susceptibility to type I diabetes in, for example, an Ashkenazi Jew or an individual or Middle European descent. Type I diabetes, also known as insulin-dependent diabetes mellitus (IDDM), usually appears in childhood but can appear at any age and is due to a deficiency of insulin, which may be caused by inadequate proinsulin production by the pancreas, by an accelerated destruction of insulin, or by insulin antagonists and inhibitors. Type I diabetes is typically characterized by polydipsia, polyuria, increased appetite, weight loss, low plasma insulin levels, and episodic ketoacidosis.

The methods of the invention can also be used to diagnose or predict susceptibility to a particular subtype of Crohn's disease in a patient. Such a subtype can be, for example, a clinical subtype of Crohn's disease with features of ulcerative colitis (U.S. Pat. No. 5,932,429); a clinical subtype of Crohn's disease having perforating, fistulizing or small bowel obstructive disease; or a clinical subtype of CD having a superior or inferior response to anti-Th1 cytokine therapy (U.S. Pat. No. 6,183,951). The skilled person understands that these and other clinical subtypes of Crohn's disease also can be diagnosed by a JW1, JW15, JW16, JW17 or JW18 variant allele or another disease-predisposing allele or disease-predisposing haplotype of the invention.

The following examples are intended to illustrate but not limit the present invention.

### Example I

### Stratification Linkage Analysis of Crohn's Disease Families Based on Three NOD2/CARD15 SNPS

This example demonstrates that one or more unidentifiable disease-predisposing alleles at the IBD1 locus contribute to Crohn's disease in the Jewish population.

### A. Selection of Study Subjects

A total of 211 Crohn's disease families (64

Ashkenazi-Jewish and 147 non-Jewish Caucasian families) consisting of 373 Crohn's disease patients and 672 unaffected relatives were studied. The probands of all families were ascertained from the IBD Center at Cedars-Sinai Medical Center or referred to Cedars-Sinai Medical Center by gastroenterologists or the Crohn's Colitis Foundation of America nationwide. These families had at least one family member affected with Crohn's disease and did not have any known individuals affected with ulcerative colitis. In these 211 families, 91 multiplex Crohn's disease families that included Crohn's disease sib-pairs were available for linkage analysis (28 Ashkenazi-Jewish and 63 non-Jewish families).

An independent case-control was panel made up of 112 Ashkenazi-Jewish and 166 non-Jewish patients with Crohn's disease, 79 Ashkenazi Jewish and 143 non-Jewish control individuals. Controls were recruited from spouses, married-in relatives, or acquaintances who had no known IBD or other autoimmune diseases or family history of IBD. The use of human subjects was reviewed and approved by the Human Subject Institutional Review Board at Cedars-Sinai Medical Center.

### B. Stratification Linkage Analysis on the NOD2/CARD15 Genotype

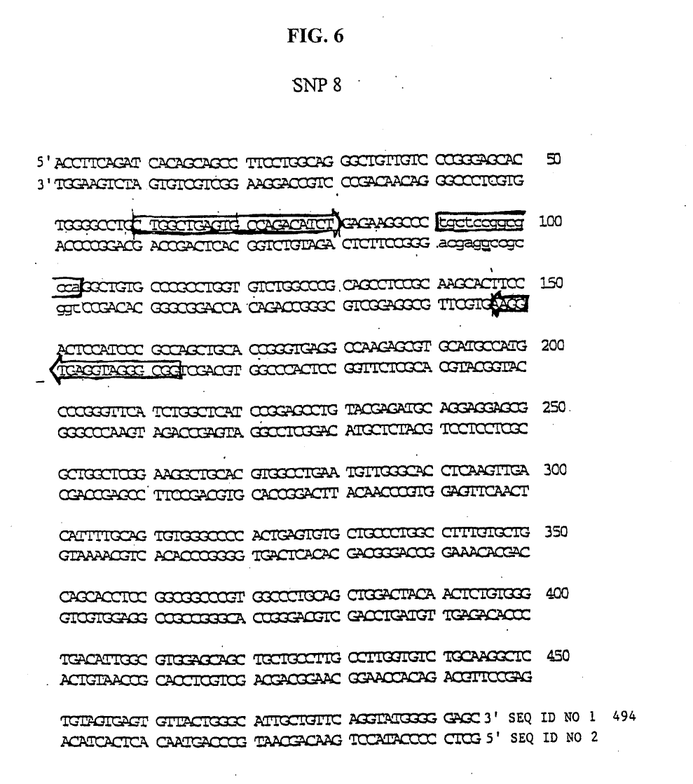

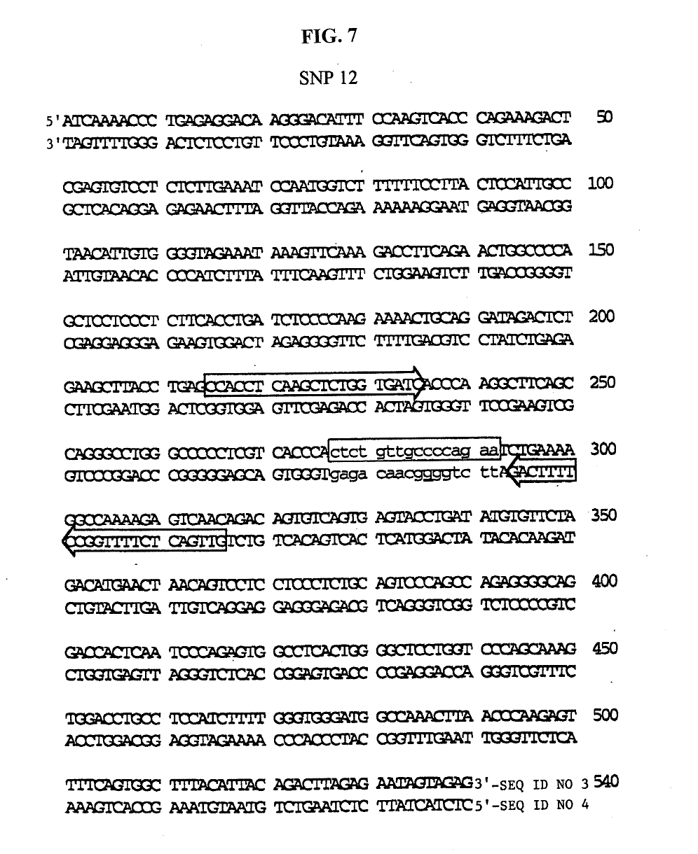

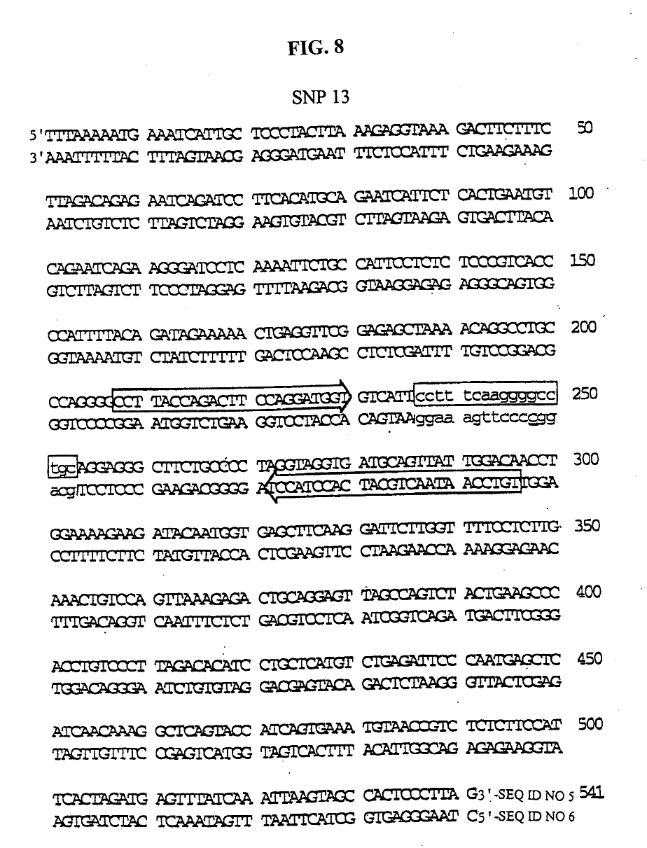

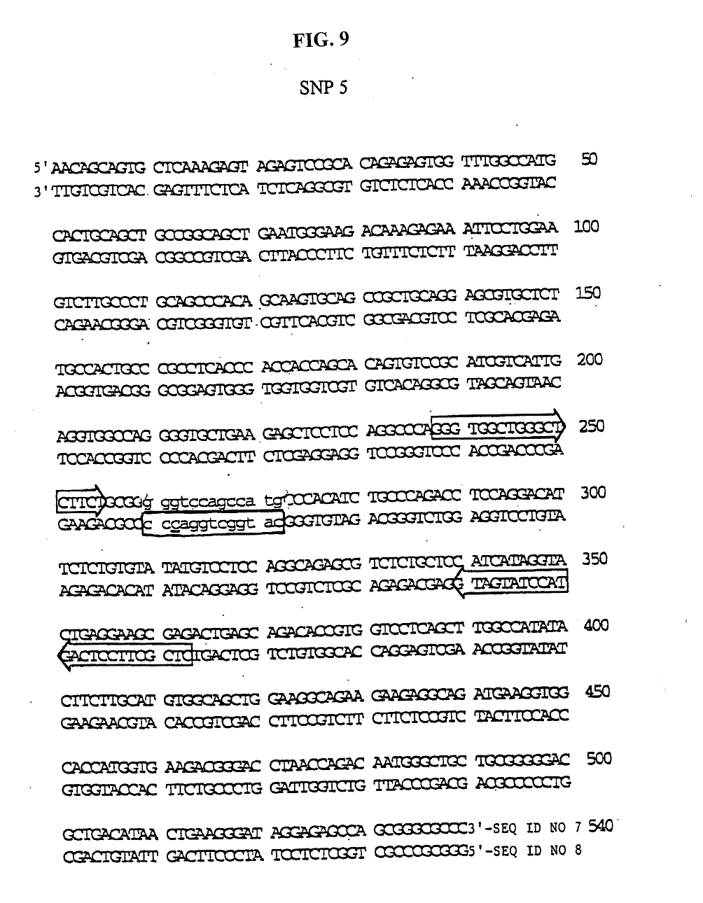

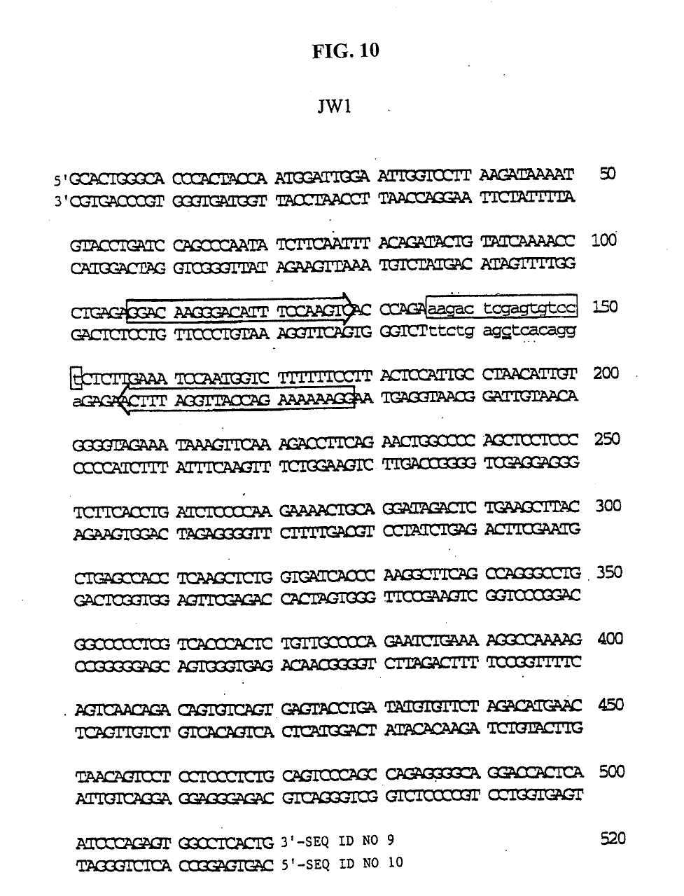

Families were genotyped for six microsatellite markers spanning the IBD1 locus, covering 34 cM on chromosome 16. The six microsatellite markers were D16S403, D16S753, D16S409, D16S411, D16S419, and D16S408. An ABI 377 automated DNA analyzer and associated software (Applied Biosystems; Foster City, Calif., USA) were used for genotype analysis. For association analysis of NOD2/CARD15, all Crohn's disease families and case-control samples were genotyped for single nucleotide polymorphic markers (SNPs) including the three principal SNPs: SNP 8 (R702W), SNP 12 (G908R), and SNP 13 (1007fs). The analyses were performed using the Taqman® MGB bialleic discrimination system (Applied Biosystems) with an ABI 7900 instrument and the probes listed in Table 1. In Table 1, 6FAM and TET are fluorescent dyes and MGBNFQ is a minor grove binder moiety. The location of the Taqman® probes on the nucleotide sequence of NOD2/CARD15 surrounding SNP8, SNP12, SNP13, the 268S allele, and the JW1 variant are shown in FIGS. 6-10, respectively.

TABLE 1
TAQMAN PROBES

| Allele | | |
|---|---|---|
| detected | Probe sequence | SIN |
| SNP5 | 6FAM-CATGGCTGGACCC-MGBNFQ | 37 |
| wild type allele | | |
| (“1”) | | |
| SNP5 | TET-CATGGCTGGATCC-MGBNFQ | 38 |
| variant allele (“2”) | | |
| SNP8 | 6FAM-TGCTCCGGCGCCA-MGBNFQ | 39 |
| wild type allele | | |
| (“1”) | | |
| SNP8 | TET-CTGCTCTGGCGCCA-MGBNFQ | 40 |
| variant allele (“2”) | | |
| SNP12 | 6FAM-CTCTGTTGCCCCAGAA- | 41 |
| wild type allele | MGBNFQ | |
| (“1”) | | |
| SNP12 | TET-CTCTGTTGCGCCAGA-MGBNFQ | 42 |
| wild type allele | | |
| (“1”) | | |
| SNP13 | TET-CTTTCAAGGGCCTGC-MGBNFQ | 43 |
| wild type allele | | |
| (“1”) | | |
| SNP13 | 6FAM-CCTTTCAAGGGGCCT- | 44 |
| variant allele (“2”) | MGBNFQ | |
| JW1 | 6FAM-AAGACTCGAGTGTCCT- | 45 |
| wild type allele | MGBNFQ | |
| JW1 | VIC-AGACTCAAGTGTCCTC- | 46 |
| variant | MGBNFQ | |

To evaluate the contribution of the three reported principal SNPs to the linkage of Crohn's disease and the IBD1 locus, all Crohn's disease multiplex families were genotyped for the three principal SNPs and then subdivided into NOD2/CARD15 SNP 8,12,13+ (positive) families, which contained at least one of the SNP8, 12 or 13 “2” alleles, and NOD2/CARD15 SNP 8,12,13− (negative) families, which contained none of the SNP8, 12 or 13 “2” alleles. Stratified linkage analysis was then performed using six microsatellite markers spanning 34 cM surrounding the NOD2/CARD15 locus on chromosome 16, based on positive or negative SNP 8, 12, and 13 “2” allele status in 35 Jewish and 70 non-Jewish sib pairs. Two-point non-parametric linkage analyses were performed using SIBPAL from S.A.G.E. version 3.1 package.

As shown in FIG. 1, in the pre-stratification analysis performed utilizing all the data in each population, a trend toward increased mean allele sharing (MAS) at markers D16S403 (31 Mb from NOD2/CARD15, MAS 0.55, p=0.11) and D16S411 (1.8 Mb from NOD2/CARD15, MAS 0.54, p=0.20) was found in the Jewish Crohn's disease families. In the non-Jewish Crohn's disease families, increased mean allele sharing (>0.50) was observed at all marker positions, with the peak at marker D16S411 (MAS 0.56, p=0.094). However, statistical significance was not found for any marker positions in the non-Jewish family sample.

When the Jewish families were divided into NOD2/CARD15 SNP 8,12,13+ and NOD2/CARD15 SNP 8,12,13-subgroups, the evidence for linkage in NOD2/CARD15 SNP 8,12,13− families increased and reached a significant level, with the two peaks at marker D16S403 (MAS=0.70, p=0.0008) at proximal 16p and at marker D16S411 (MAS=0.59, p=0.1) at proximal 16q near NOD2/CARD15 (see FIG. 1). In contrast, in the non-Jewish Crohn's disease families, significantly increased mean allele sharing was only observed in NOD2/CARD15 SNP 8,12,13+ families, with the highest mean allele sharing equal to 0.61 at D165411 (p=0.0075) while there was no evidence for increased mean allele sharing in NOD2/CARD15 SNP 8,12,13− families (MAS=0.47 to 0.52). These results indicated a different contribution of the three known single nucleotide polymorphic markers, SNP8, SNP12, and SNP13, to the IBD1 linkage of Crohn's disease between individuals of Ashkenazi Jewish descent and other Caucasians.

Furthermore, significant linkage still remained after stratification in the Jewish Crohn's disease families which did not carry any of the three principal NOD2/CARD15 SNPs. These results indicate that one or more additional predisposing genes or additional NOD2/CARD15 SNPs at the IBD1 locus play a more important role in susceptibility to Crohn's disease in the Jewish population than in the non-Jewish population.

### Example II

### Association of NOD2/CARD15 Single Nucleotide Polymorphisms with Crohn's Disease

This example describes the association of NOD2/CARD15 SNPs with Crohn's disease in an Ashkenazi Jewish population.

### A. Transmission Disequilibrium Test (TDT)

A family based association test was performed using a transmission disequilibrium test (TDT; Spielman et al., “Transmission test for linkage disequilibrium: the insulin gene region and insulin-dependent diabetes mellitus (IDDM),” Am. J. Hum. Genet. 52:506-516 (1993)) to investigate any ethnic based differences of Crohn's disease association by the three principal NOD2/CARD15 SNPs. For this analysis, the 64 Jewish and 147 non-Jewish families were used as the sample. The analysis confirmed that the SNP8, SNP12, and SNP13 variants almost always occur on a common background haplotype which includes the 268S allele (“286S alone haplotype”), and that the SNP8, SNP12, and SNP13 variants were never found on the same haplotype in both family groups.

As shown in Table 2, in the non-Jewish families, there were very significant associations with the 268S/702W haplotype (T/NT: 42/11, p=0.000021) and the 268S/1007fs haplotype (T/NT: 34/12, p=0.0012). There was also an increased frequency of 268S/908R haplotype transmission (T/NT: 10/6), although this was not statistically significant. In contrast, when a moderate number of Jewish families was analyzed, a significant association between Crohn's disease and the 268S/908R haplotype (T/NT: 13/2, p=0.0045) was observed while the association of the 268S/1007fs and 268S/702W haplotypes was not observed (See Table 2). In addition, there appeared to be a difference between these two ethnic groups in the distribution of the haplotype containing only the 268S variant and neither the SNP8 2 allele, SNP12 2 allele, or SNP13 2 allele (“268S alone” haplotype). As shown in Table 2, preferential transmission of the haplotype with only the background variant allele (268S) was found in Jewish families (T/NT: 8/5), but not in non-Jewish families (T/NT: 31/38). To perform the TDT, GENEHUNTER2 was used for four loci SNP haplotypes (Kruglyak et al., “Parametric and nonparametric linkage analysis: a unified multipoint approach,” Am. J. Hum. Genet. 58:1347-1363 (1996)), and SIMWALKER2 was used to construct haplotypes for five SNPs (Sobel and Lange, “Descent graphs in pedigree analysis: applications to haplotyping, location scores, and marker-sharing statistics,” Am. J. Hum. Genet. 58:1323-1337 (1996)). Each of the haplotypes was tested for its transmission distortion using the family based TDT method.

TABLE 2
Transmission disequilibrium test (TDT) of the NOD2/CARD15
haplotypes in Ashkenazi-Jewish and non-Jewish Caucasian
families with Crohn's disease

| | |
|---|---|
| Ashkenazi Jews | Non-Jewish Caucasians |

| Haplotype^{a} | T/NT^{b} | p-value | T/NT^{b} | p-value |
|---|---|---|---|---|
| 702W | 2/4 | | 42/11 | 0.000021 |
| 908R | 13/2 | 0.0045 | 10/6 | |
| 1007fs | 5/4 | | 34/12 | 0.0012 |
| 268S alone | 8/5 | | 31/38 | |
| No variant | 16/27 | | 36/85 | |

^{a}The haplotypes 702W, 908R, and 1007fs represent the haplotype which has the rare variant of each mutation. All 702W, 908R, and 1007fs haplotypes also possess the rare variant of the background SNP (268S).
^{b}Transmitted/Not Transmitted

### B. Case Control Study

The negative association of certain SNPs in Jewish families and preferential transmission of the “268S alone” haplotype was further tested in a larger ethnically matched case-control sample. Due to small numbers in rare allele homozygotes and compound heterozygotes, the rare allele homozygotes were combined with heterozygotes, and all other rare haplotypes were separately combined so that six main haplotypic groups were formed as indicated in Table 3. Since only 29% of the families were multiplex families and there was no difference in the transmission disequilibrium test results between simplex and multiplex families, all families were combined. The case-control study was performed using PHASE to construct haplotypes (Stephens et al., “A new statistical method for haplotype reconstruction from population data,” Am. J. Hum. Genet. 68:978-989 (2001)). Haplotypic genotype frequencies were compared between cases and controls using the chi-squared test.

Using comparable sample sizes, a significant association between the haplotype with the 268S allele and the frame shift mutation (1007fs; 2112 haplotype) and Crohn's disease was observed in both the Jewish (OR=7.50, p=0.0041) and non-Jewish samples (OR=3.54, p=0.024) (combined p<0.001). Increases of the haplotype including the 268S allele and 908R (2121 haplotype) were seen in both ethnic groups, though these did not attain statistical significance possibly due to the sample size. A significant association with Crohn's disease in the non-Jewish population also was observed with regard to the 268S and 702W haplotype (2211 haplotype; OR=2.50, p=0.022). In the Jewish population, the association of the 702W haplotype did not reach statistical significance, though an increased odds ratio was observed (OR=2.00, p=0.24).

A highly significant association also was observed between the haplotype carrying only the background variation (“268S alone” or “2111” haplotype) and Crohn's disease in the Jewish sample population. The association between the 268S alone haplotype with Crohn's disease in the Jewish population had the lowest p value (p=0.0023, OR=3.13) when compared with any of the other three haplotypes analyzed. No evidence for association of the “268S alone” (2111) haplotype was found in non-Jews (p=0.834, OR=1.06). These results indicate that the 268S alone haplotype is associated with susceptibility to Crohn's disease in individuals of Ashkenazi Jewish descent. These results further indicate that the 268S alone haplotype can contain one or more unrecognized predisposing mutations (as described further below).

TABLE 3
Association studies of NOD2/CARD15 haplotype groups defined by four SNPs in Ashkenazi-Jewish and
non-Jewish Caucasian patients with Crohn's disease

| 4 SNP | Ashkenazi Jews | | | | | Non-Jewish Caucasians | | | | |
|---|---|---|---|---|---|---|---|---|---|---|
| loci | CD^{b} | control | | | | CD^{b} | control | | | |
| Haplotype^{a} | (%) | (%) | p-value | OR^{c} | 95% CI^{d} | (%) | (%) | p-value | OR^{c} | 95% CI^{d} |
| 702W | 7.1 | 6.3 | 0.25 | 2.00 | 0.6-6.6 | 13.8 | 6.9 | 0.022 | 2.50 | 1.1-5.6 |
| 908R | 12.5 | 8.8 | 0.067 | 2.50 | 0.9-6.8 | 5.4 | 3.5 | 0.24 | 1.96 | 0.6-6.1 |
| 1007fs | 10.7 | 2.5 | 0.0041 | 7.50 | 1.6-35.5 | 7.8 | 2.8 | 0.025 | 3.54 | 1.1-11.3 |
| 268S alone | 31.2 | 17.7 | 0.0023 | 3.13 | 1.5-6.6 | 22.2 | 26.5 | 0.83 | 1.06 | 0.6-1.8 |
| Other | 2.6 | 1.2 | 0.23 | 3.75 | 0.4-37.4 | 3.0 | 0 | 0.022 | | |
| Reference | 35.7 | 63.2 | | | | 47.5 | 60.1 | | | |

^{a}Each haplotype group in ‘4 SNP haplotype’ consists of individuals with the following haplotypic genotype. Each SNP position is P268S, R702W, G908R, and 1007fs respectively. ‘1’ is wild type, ‘2’ is the rare variant of the SNP. 702W: 2211/1111, 2211/2211, 2211/2211, 1211/1111. 908R: 2121/1111, 2121/2111. 1007fs: 2112/1111, 2112/2111, 2112/2112. Other: 2112/2211, 2121/2211, 2112/2121, 2121/1211. 268S alone: 21111/2111, 2111/1111. Reference haplotypic genotype: 1111/1111.
^{b}Crohn's disease.
^{c}Odds ratio.
^{d}Confidence interval.

### Example III

### Identification of Novel NOD2/CARD15 Disease Predisposing Mutations in Individuals of Ashkenazi Jewish Descent

This example describes identification of twelve NOD2/CARD15 sequence variants in individuals of Ashkenazi Jewish descent.

To search for disease predisposing mutations on the 268S alone haplotype in the Jewish population, NOD2/CARD15 genes, including exons, 5′ untranslated regions (UTR) and splicing signal regions, were sequenced from twelve Jewish individuals. Genomic DNA was obtained from twelve individuals of Ashkenazi descent. These individuals consisted of seven Crohn's disease patients with the 268S alone haplotype (CD1-CD7 in FIG. 2), and three patients (CD8-CD10) and two normal controls (NC1 and NC2) without the 268S variant allele. As shown in FIG. 2, 12 sequence variants in NOD2/CARD15 were identified. One of these, an intervening sequence variant, IVS8+158, is a C to T mutation in the palindrome sequence within intron 8 and was designated “JW1”. Another, R791Q, is a G to A mutation which results in the substitution of arginine to glutamine within exon 4. Non-silent coding region mutations were not common in Jewish Crohn's disease patients with the 268S alone haplotype.

FIG. 2 shows the location of each of the variants in the NOD2/CARD15 gene and the profile of variants found in each of the 12 sequenced individuals. Of the 12, nine variants were found in Jewish patients with the 268S alone haplotype. The most common variant observed in those patients was IVS10-133 (SNP9). In addition, 5′UTR-59 (rs2076752), S178S (rs2067085), R459R (SNP6), R587R (SNP7), R791Q, IVS8+158 (JW1), V955I, and IVS10+64 (rs1077861) were also observed.

Genomic DNA was isolated from immortalized cell lines derived from patient lymphocytes isolated from a whole blood sample taken from each patient following informed consent. Immortalized cell lines were made by transforming lymphocytes using Epstein-Barr virus using methods adapted from Anderson and Gusella, In Vitro 20:856-858 (1984), Miller and Lipman, Proc. Natl. Acad. Sci. USA 70:190-194 (1973), Freshney, A Manual of Basic Techniques. 2^{nd} ed. New York: Alan R. Liss (1987), and Pressman and Rotter, Am. J. Hum. Genet. 49:467 (1991).

Immortalized cell lines were grown, and aliquots of 5 million cells were frozen in liquid nitrogen for future use. Genomic DNA was then isolated from one of the aliquots using extraction by phenol-chloroform and ethanol precipitation following the procedures of Herrman and Frischauf, Isolation of genomic DNA. Methods in Enzymology 152:180-183 (1987), and Sambrook et al., Molecular Cloning. New York: Cold Spring Harbor Laboratory Press (1989).

DNA sequencing was performed according using the Big Dye Terminator Ready Reaction kit according to the manufacturer's instructions (Applied Biosystems). The sequencing primers are shown in Table 2 and were identical to primers used for PCR, except that the nested primers were used for sequencing exon 9. Sequence data were analyzed on an ABI 377 DNA analyzer with associated software. Alignments were performed using CLUSTALW (Thompson et al., Nucleic Acids Res. 22:4673-4680 (1994)).

TABLE 4

| Primers used for sequencin NOD2/CARD15 | | | | | | |
|---|---|---|---|---|---|---|
| | | SEQ | | SEQ | | Annl. |
| | | ID | | ID | Size | Temp. |
| | Forward Primer | NO. | Reverse Primer | NO. | (BP) | (° C.) |
| Exl | TCTCCTCCCCAGATGTTTAAGATG | 47 | CCAGCCAAGGATGCCACAGC | 48 | 856 | 63 |
| Ex2 | TGCCTTCTCTGGGTCTCAAT | 49 | ATGGACCAAGTTACCCCACA | 50 | 751 | 53 |
| Ex3 | GACTGCCCTTCCCTTTCTG | 51 | ACATTGCTCCATCAGCCTTC | 52 | 200 | 55 |
| Ex4D | CAGAGCCCCTTCCCGTCATC | 53 | AGCACAGTGTCCGCATCGTCATTG | 54 | 625 | 65 |
| Ex4C | CTGGAGGAGCTCTTCAGCAC | 55 | AACAGTTCCTGGTGGCATTT | 56 | 668 | 60 |
| Ex4B | CCTGCTCCAAGAGACCTCAG | 57 | TCAGATGTCTGGCACTCAGC | 58 | 678 | 60 |
| Ex4A | AGATCACAGCAGCCTTCCTG | 59 | ATCTGGGCAGTGTTGCAAAG | 60 | 582 | 60 |
| Ex5&6 | TTTTGGGGGATTTGTAGATT | 61 | CTGGGGAGATCACAGCATTAGAGA | 62 | 631 | 54 |
| Ex7 | ACTCTCTCCCTGGCTTGTC | 63 | CGTCCCGCTGCCCCTTTC | 64 | 435 | 55 |
| Ex8 | GAGGCCACTCTGGGATTGAG | 65 | CCTGATCCAGCCCAATATCTT | 66 | 463 | 57 |
| Ex9 | TGCCAGGCACTATATTAAGGT | 67 | GGGCTGGATCAGGTACATT | 68 | 878 | 53 |
| Ex10 | CTTTATTGGTTACCTTCACTTC | 69 | GCTGCAATGGAGAGTGGG | 70 | 654 | 55 |
| Exll | GATGGCACGGGTACTCTT | 71 | ACTGAGGTTCGGAGAGCTAAA | 72 | 511 | 56 |
| Ex12 | GAGGGCACCAGGGTTTGCTCA | 73 | GATCAGCAGAGGCCAGTCCCATAC | 74 | 698 | 56 |
| Ex9 | CCCCAGAGCAGAGAATCC | 75 | CTTTCCCTGCTCTGACATAC | 76 | | 55 |
| nested | | | | | | |

### Example IV

### Linkage Disequilibrium of JW1 with Other SNPs

This example demonstrates that JW1 is in linkage disequilibrium with other NOD2/CARD15 single nucleotide polymorphic markers.

The newly identified IVS8+158 (JW1) variant was identified as an allelic variant in Crohn's disease patients with the 268S alone (2111) haplotype (see FIG. 2). In view of this discovery, individuals of Ashkenazi Jewish descent were genotyped for the JW1 variant as a possible disease-predisposing mutation or as a marker identifying a disease-predisposing haplotype. As described above, since the available sample size in the Jewish families was limited for analysis of the 268S alone haplotype variation, the case-control panel was utilized to investigate the role of the JW1 variant.

Results obtained with the case-control panel showed that the JW1 variant was in linkage disequilibrium with other NOD2/CARD15 SNPs. In the Jewish population, all of the 1007fs and 702W variants occurred on the same haplotype as JW1. In contrast, the 908R variant occurred on the haplotype that did not possess JW1 (FIG. 3). This trend was similarly observed in the non-Jewish groups. These results indicate that, in the Jewish population, the JW1 variant is in linkage disequilibrium with several single nucleotide polymorphic markers associated with Crohn's disease.

### Example V

### Association of the 268S-JW1 Haplotype with Crohn's Disease

This example demonstrates that the 268S-JW1 haplotype is associated with Crohn's disease in individuals of Ashkenazi Jewish descent.

The 268S alone haplotype was divided into two haplotype groups based on the presence or absence of the JW1 variant in each ethnic group. As shown in Table 5, in this haplotypic genotype analysis, a strong association of the 268S-JW1 haplotype with Crohn's disease was observed in the Jewish population independent of the other SNP haplotype groups.

As compared to the 268S alone haplotype group, the 268S-JW1 haplotype showed a remarkably increased association (OR=5.75), and the most significant p value (p=0.0005) among all haplotype groups in the Jewish population. Furthermore, as shown in FIG. 5, the 268S-JW1 group showed the highest population attributable risk (PAR, 15.1%) among all haplotypes for individuals of Ashkenazi Jewish descent. When the 268S-JW1 haplotype was taken into account, the total population attributable risk of NOD2/CARD15 risk variants in Ashkenazi Jews was 28.3%, which was double the population attributable risk of the three principal SNPs observed in non-Jewish Caucasians (14.6%).

In summary, these results demonstrate that, in contrast to results obtained in the Ashkenazi-Jewish population, in the non-Jewish population, no association was found between Crohn's disease and either the 268S-JW1(+) haplotype (CD 15.6% vs. control 15.3%) or the 268S-JW1(−) haplotype (6.6% vs. 11.1%).

The odds ratio (OR) and its 95% confidence interval (95% CI) were calculated for each risk haplotype by the Mantel-Haenszel method (Schlesselman, J J. Case-Control Studies Design, Conduct, Analysis. Oxford University Press, New York, pp. 183-190 (1982)). Population attributable risk was estimated assuming that the frequency of a risk haplotype in the control group can be regarded as approximately representative of the target population and using the odds ratio as an approximation of the relative risk. The population attributable risk was calculated as PAR=Pe(OR−1)/[Pe(OR−1)+1], where Pe=frequency of a risk haplotype in control group and OR=odds ratio (Schlesselman, supra, 1982).

TABLE 5
Association studies of NOD2/CARD15 haplotype groups defined by JW1 in Ashkenazi-Jewish and
non-Jewish Caucasian patients with Crohn's disease

| 5 SNP | Ashkenazi Jews | | | | | Non-Jewish Caucasians | | | | |
|---|---|---|---|---|---|---|---|---|---|---|
| loci | CD^{b} | Control | | | | CD^{b} | Control | | | |
| Haplotype^{a} | (%) | (%) | p-value | OR^{c} | 95% CI^{d} | (%) | (%) | p-value | OR^{c} | 95% CI^{d} |
| 268S-JW1 | 20.5 | 6.3 | 0.0005 | 5.75 | 2.0-16.4 | 15.6 | 15.3 | 0.4431 | 1.29 | 0.7-2.5 |
| 268S-JW1(1) | 10.7 | 11.3 | 0.29 | 1.67 | 0.6-4.3 | 6.6 | 11.1 | 0.4908 | 0.75 | 0.3-1.7 |

Note:
As ‘Reference’, ‘702W’, ‘908R’, ‘1007fs’, and ‘Other’ haplotypic genotype group showed the same values as in Table 3, those were omitted in this table.
^{a}Each haplotype group in ‘5 SNP haplotype’ consists of individuals with the following haplotypic genotype. Each SNP position is P268S, R702W, G908R, JW1 and 1007fs respectively. ‘1’ means wild type, ‘2’ is the rare variant of the SNP. 268S-JW1 (without other three DPMs): 21121/11111, 21121/21111, 21121/21121, 21121/11121. 268S-JW1(—) (without other three DPMs): 11111/21111, 21111/21111.
^{b}Crohn's disease.
^{c}Odds ratio.
^{d}Confidence interval.

### Example VI

### Identification of NOD2/CARD15 Sequence Variants JW15, JW16, JW17, and JW18

This example describes identification of several allelic variants associated with Crohn's disease.

Sequence analysis of individuals with the 268S-JW1 haplotype led to the identification of additional sequence variants. Two sequence variants, designated JW17 and JW18, were identified in the NOD2/CARD15 5′ untranslated region. The position of these sequence variants is shown in FIG. 11. In relation to the AC007728 sequence, the JW17 variant is a cytosine to thymine change located at position 154,688 and the JW18 variant is a cytosine to thymine change located at position 154,471.

Nucleotides 153,601 to 155,000 of the AC007728 sequence were submitted to the Transcription Element Search System (TESS) (see Schug and Overton, Technical Report CBIL-TR-1997-1001-v0.0 of the Computational Biology and Information Laboratory, School of Medicine, University of Pennsylvania, (1997)). The JW17 site is nucleotide 01088 in the TESS submission, and the JW18 site is nucleotide 871 in the TESS submission. JW17 and JW18 are located in regions of the NOD2/CARD15 5′ untranslated region that contain binding sites for transcription factors. For example, the JW18 variant is a cytosine to thymine change that alters the sequence in a binding site for Oct-1 and Zeste transcription factors, and the JW17 variant is a cytosine to thymine change that alters the sequence in a binding site immediately adjacent to a PHO4 binding site. Both changes can result in alterations in the regulation of NOD2/CARD15 by affecting binding of transcription factors and, thus, transcription from the NOD2/CARD15 promoter region.

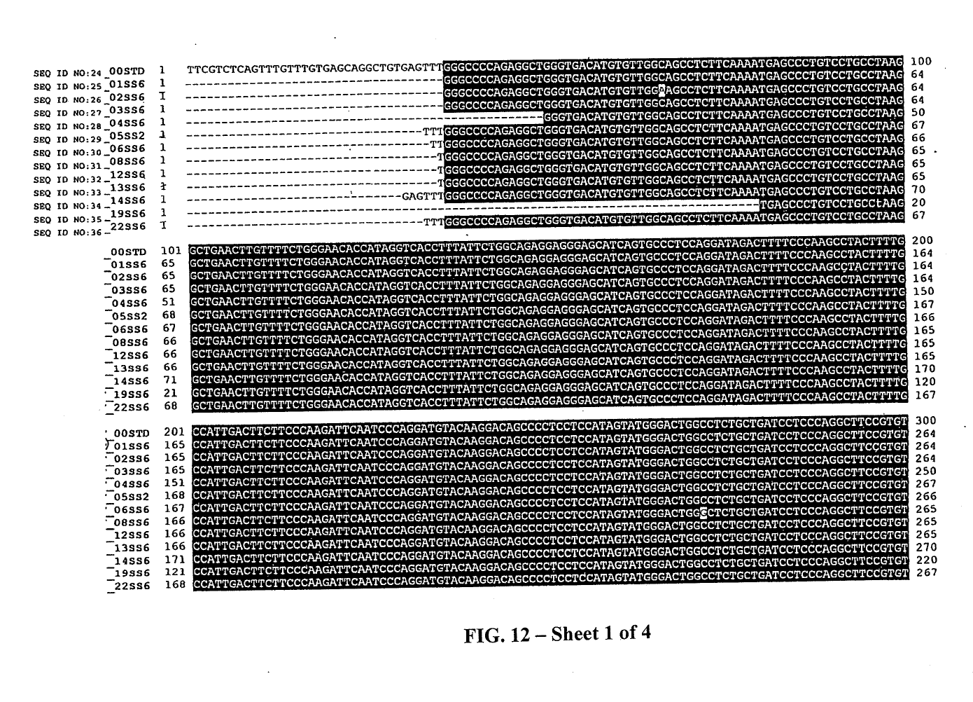

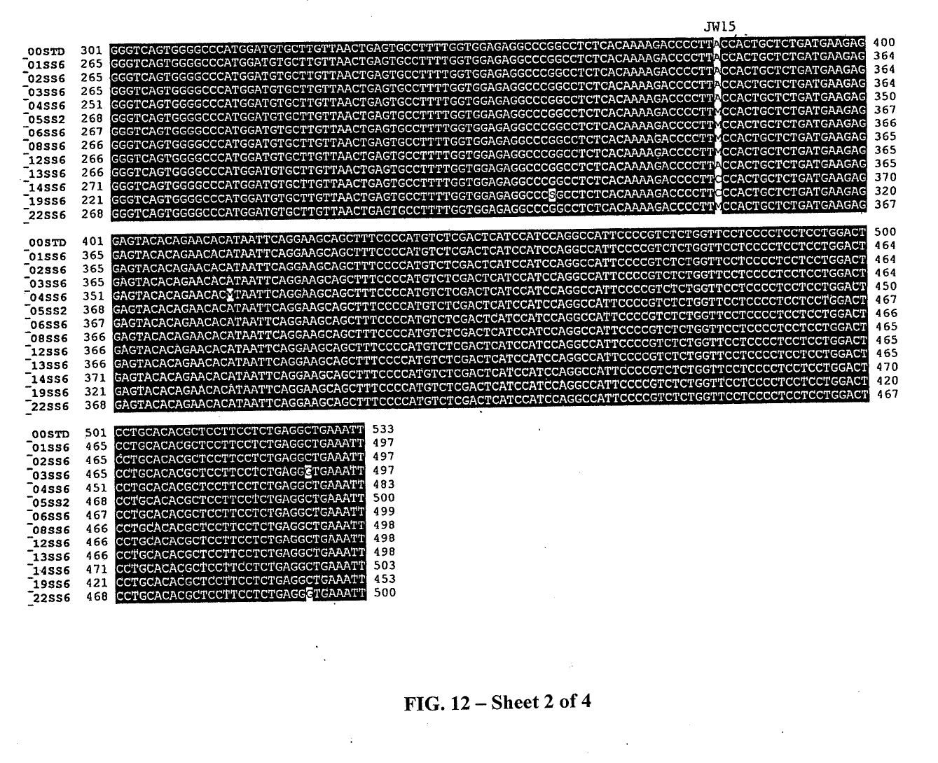

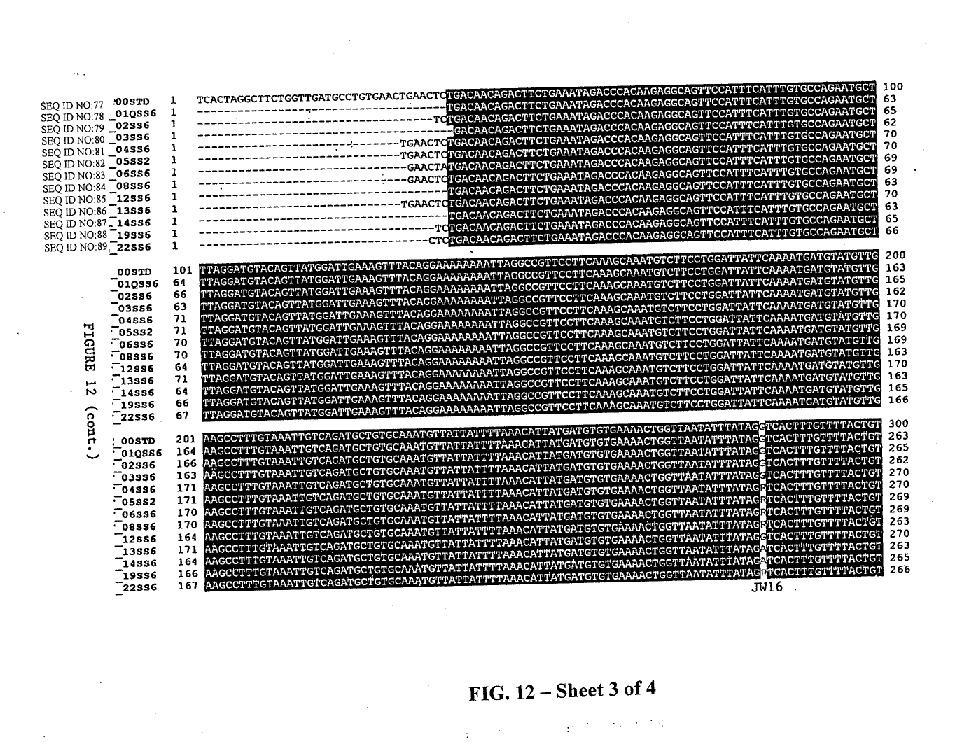

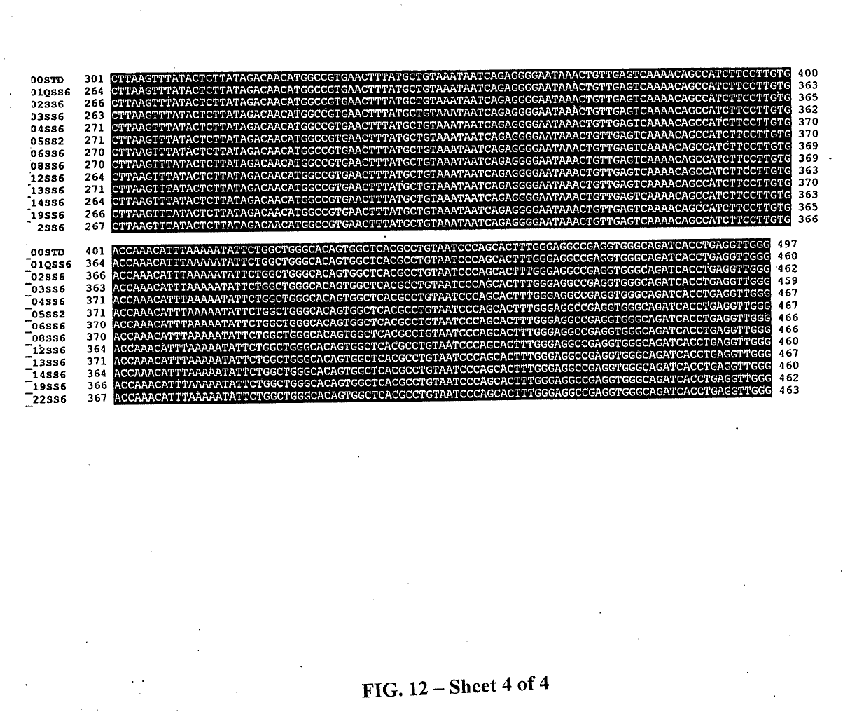

In addition to variants identified in the NOD2/CARD15 5′ untranslated region, two variants, designated JW15 and JW16, were identified in the NOD2/CARD15 3′ untranslated region. The position of these sequence variants is shown in FIG. 12. In relation to the AC007728 sequence, the JW15 site is located at position 118,790, and the JW16 site is located at position 118,031. These allelic variants can result, for example, in alteration of NOD2/CARD15 mRNA stability or alteration in the binding of proteins to the 3′ untranslated region of NOD2/CARD15, thus, affecting the amount of NOD2/CARD2 protein produced.

All journal article, reference, and patent citations provided above, in parentheses or otherwise, whether previously stated or not, are incorporated herein by reference.

Although the invention has been described with reference to the examples above, it should be understood that various modifications can be made without departing from the spirit of the invention. Accordingly, the invention is limited only by the following claims.

1. A method of diagnosing or predicting susceptibility to Crohn's disease in an individual, comprising determining the presence or absence in said individual of a disease-predisposing haplotype comprising a JW1 variant allele at the NOD2/CARD15 locus,
wherein the presence of said disease-predisposing haplotype is diagnostic of or predictive of susceptibility to Crohn's disease.

2-14. (canceled)

15. A method of diagnosing or predicting susceptibility to Crohn's disease in an individual, comprising determining the presence or absence in said individual of a disease-predisposing haplotype comprising a 268S allele and a JW1 variant allele at the NOD2/CARD15 locus,
wherein the presence of said disease-predisposing haplotype is diagnostic of or predictive of susceptibility to Crohn's disease.

16-28. (canceled)

29. A method of diagnosing or predicting susceptibility to Crohn's disease in an individual, comprising determining the presence or absence in said individual of a JW1 variant allele at the NOD2/CARD15 locus,
wherein the presence of said JW1 variant allele is diagnostic of or predictive of susceptibility to Crohn's disease.

38. A method of diagnosing or predicting susceptibility to Crohn's disease in an individual, comprising determining the presence or absence in said individual of a disease-predisposing allele linked to a JW1 variant allele at the NOD2/CARD15 locus, provided that when said disease-predisposing allele is combined in a haplotype with a 268S allele, said haplotype is associated with Crohn's disease in an Ashkenazi Jewish population with a PAR value of at least 9,
wherein the presence of said disease-predisposing allele is diagnostic of or predictive of susceptibility to Crohn's disease.

39-54. (canceled)


SEQUENCE LISTING (89 sequences)

SEQ ID NO: 1 - DNA, 494 bp, Homo sapiens
  nucleotide sequence of NOD2/CAD15 surrounding SNP 8 top strand
accttcagatcacagcagccttcctggcagggctgttgtcccgggagcactggggcctgctggctgagtgccagacatctgagaaggccctgctccggcgccaggctgtgcccgcctggtgtctggcccgcagcctccgcaagcacttccactccatcccgccagctgcaccgggtgaggccaagagcgtgcatgccatgcccgggttcatctggctcatccggagcctgtacgagatgcaggaggagcggctggctcggaaggctgcacgtggcctgaatgttgggcacctcaagttgacattttgcagtgtgggccccactgagtgtgctgccctggcctttgtgctgcagcacctccggcggcccgtggccctgcagctggactacaactctgtgggtgacattggcgtggagcagctgctgccttgccttggtgtctgcaaggctctgtagtgagtgttactgggcattgctgttcaggtatgggggagc

SEQ ID NO: 2 - DNA, 494 bp, Homo sapiens
  nucleotide sequence of NOD2/CAD15 surrounding SNP 8 bottom strand
gctcccccatacctgaacagcaatgcccagtaacactcactacagagccttgcagacaccaaggcaaggcagcagctgctccacgccaatgtcacccacagagttgtagtccagctgcagggccacgggccgccggaggtgctgcagcacaaaggccagggcagcacactcagtggggcccacactgcaaaatgtcaacttgaggtgcccaacattcaggccacgtgcagccttccgagccagccgctcctcctgcatctcgtacaggctccggatgagccagatgaacccgggcatggcatgcacgctcttggcctcacccggtgcagctggcgggatggagtggaagtgcttgcggaggctgcgggccagacaccaggcgggcacagcctggcgccggagcagggccttctcagatgtctggcactcagccagcaggccccagtgctcccgggacaacagccctgccaggaaggctgctgtgatctgaaggt

SEQ ID NO: 3 - DNA, 540 bp, Homo sapiens
  nucleotide sequence of NOD2/CAD15 surrounding SNP 12 top strand
atcaaaaccctgagaggacaagggacatttccaagtcacccagaaagactcgagtgtcctctcttgaaatccaatggtcttttttccttactccattgcctaacattgtggggtagaaataaagttcaaagaccttcagaactggccccagctcctccctcttcacctgatctccccaagaaaactgcaggatagactctgaagcttacctgagccacctcaagctctggtgatcacccaaggcttcagccagggcctgggccccctcgtcacccactctgttgccccagaatctgaaaaggccaaaagagtcaacagacagtgtcagtgagtacctgatatgtgttctagacatgaactaacagtcctcctccctctgcagtcccagccagaggggcaggaccactcaatcccagagtggcctcactggggctcctggtcccagcaaagtggacctgcctccatcttttgggtgggatggccaaacttaacccaagagttttcagtggctttacattacagacttagagaatagtagag

SEQ ID NO: 4 - DNA, 540 bp, Homo sapiens
  nucleotide sequence of NOD2/CAD15 surrounding SNP 12 bottom strand
ctctactattctctaagtctgtaatgtaaagccactgaaaactcttgggttaagtttggccatcccacccaaaagatggaggcaggtccactttgctgggaccaggagccccagtgaggccactctgggattgagtggtcctgcccctctggctgggactgcagagggaggaggactgttagttcatgtctagaacacatatcaggtactcactgacactgtctgttgactcttttggccttttcagattctggggcaacagagtgggtgacgagggggcccaggccctggctgaagccttgggtgatcaccagagcttgaggtggctcaggtaagcttcagagtctatcctgcagttttcttggggagatcaggtgaagagggaggagctggggccagttctgaaggtctttgaactttatttctaccccacaatgttaggcaatggagtaaggaaaaaagaccattggatttcaagagaggacactcgagtctttctgggtgacttggaaatgtcccttgtcctctcagggttttgat

SEQ ID NO: 5 - DNA, 541 bp, Homo sapiens
  nucleotide sequence of NOD2/CAD15 surrounding SNP 13 top strand
tttaaaaatgaaatcattgctccctacttaaagaggtaaagacttctttcttagacagagaatcagatccttcacatgcagaatcattctcactgaatgtcagaatcagaagggatcctcaaaattctgccattcctctctcccgtcaccccattttacagatagaaaaactgaggttcggagagctaaaacaggcctgcccaggggccttaccagacttccaggatggtgtcattcctttcaaggggcctgcaggagggcttctgcccctaggtaggtgatgcagttattggacaacctggaaaagaagatacaatggtgagcttcaaggattcttggttttcctcttgaaactgtccagttaaagagactgcaggagttagccagtctactgaagcccacctgtcccttagacacatcctgctcatgtctgagattcccaatgagctcatcaacaaaggctcagtaccatcagtgaaatgtaaccgtctctcttccattcactagatgagtttatcaaattaagtagccactcccttag

SEQ ID NO: 6 - DNA, 541 bp, Homo sapiens
  nucleotide sequence of NOD2/CAD15 surrounding SNP 13 bottom strand
ctaagggagtggctacttaatttgataaactcatctagtgaatggaagagagacggttacatttcactgatggtactgagcctttgttgatgagctcattgggaatctcagacatgagcaggatgtgtctaagggacaggtgggcttcagtagactggctaactcctgcagtctctttaactggacagtttcaagaggaaaaccaagaatccttgaagctcaccattgtatcttcttttccaggttgtccaataactgcatcacctacctaggggcagaagccctcctgcaggccccttgaaaggaatgacaccatcctggaagtctggtaaggcccctgggcaggcctgttttagctctccgaacctcagtttttctatctgtaaaatggggtgacgggagagaggaatggcagaattttgaggatcccttctgattctgacattcagtgagaatgattctgcatgtgaaggatctgattctctgtctaagaaagaagtctttacctctttaagtagggagcaatgatttcatttttaaa

SEQ ID NO: 7 - DNA, 540 bp, Homo sapiens
  nucleotide sequence of NOD2/CAD15 surrounding SNP 5 top strand
aacagcagtgctcaaagagtagagtccgcacagagagtggtttggccatgcactgcagctgccggcagctgaatgggaagacaaagagaaattcctggaagtcttgccctgcagcccacagcaagtgcagccgctgcaggagcgtgctcttgccactgcccgcctcacccaccaccagcacagtgtccgcatcgtcattgaggtggccaggggtgctgaagagctcctccaggcccagggtggctgggctcttctgcgggggtccagccatgcccacatctgcccagacctccaggacattctctgtgtatatgtcctccaggcagagcgtctctgctccatcataggtactgaggaagcgagactgagcagacaccgtggtcctcagcttggccatatacttcttgcatgtggcagctggaaggcagaagaagaggcagatgaaggtggcaccatggtgaagacgggacctaaccagacaatgggctgctgcgggggacgctgacataactgaagggataggagagccagcgggcgccc

SEQ ID NO: 8 - DNA, 540 bp, Homo sapiens
  nucleotide sequence of NOD2/CAD15 surrounding SNP 5 bottom strand
gggcgcccgctggctctcctatcccttcagttatgtcagcgtcccccgcagcagcccattgtctggttaggtcccgtcttcaccatggtgccaccttcatctgcctcttcttctgccttccagctgccacatgcaagaagtatatggccaagctgaggaccacggtgtctgctcagtctcgcttcctcagtacctatgatggagcagagacgctctgcctggaggacatatacacagagaatgtcctggaggtctgggcagatgtgggcatggctggacccccgcagaagagcccagccaccctgggcctggaggagctcttcagcacccctggccacctcaatgacgatgcggacactgtgctggtggtgggtgaggcgggcagtggcaagagcacgctcctgcagcggctgcacttgctgtgggctgcagggcaagacttccaggaatttctctttgtcttcccattcagctgccggcagctgcagtgcatggccaaaccactctctgtgcggactctactctttgagcactgctgtt

SEQ ID NO: 9 - DNA, 520 bp, Homo sapiens
  nucleotide sequence of NOD2/CAD15 surrounding JW1 variant top strand
gcactgggcacccactaccaatggattggaattggtccttaagataaaatgtacctgatccagcccaatatcttcaatttacagatactgtatcaaaaccctgagaggacaagggacatttccaagtcacccagaaagactcgagtgtcctctcttgaaatccaatggtcttttttccttactccattgcctaacattgtggggtagaaataaagttcaaagaccttcagaactggccccagctcctccctcttcacctgatctccccaagaaaactgcaggatagactctgaagcttacctgagccacctcaagctctggtgatcacccaaggcttcagccagggcctgggccccctcgtcacccactctgttgccccagaatctgaaaaggccaaaagagtcaacagacagtgtcagtgagtacctgatatgtgttctagacatgaactaacagtcctcctccctctgcagtcccagccagaggggcaggaccactcaatcccagagtggcctcactg

SEQ ID NO: 10 - DNA, 520 bp, Homo sapiens
  nucleotide sequence of NOD2/CAD15 surrounding JW1 variant bottom strand
cagtgaggccactctgggattgagtggtcctgcccctctggctgggactgcagagggaggaggactgttagttcatgtctagaacacatatcaggtactcactgacactgtctgttgactcttttggccttttcagattctggggcaacagagtgggtgacgagggggcccaggccctggctgaagccttgggtgatcaccagagcttgaggtggctcaggtaagcttcagagtctatcctgcagttttcttggggagatcaggtgaagagggaggagctggggccagttctgaaggtctttgaactttatttctaccccacaatgttaggcaatggagtaaggaaaaaagaccattggatttcaagagaggacactcgagtctttctgggtgacttggaaatgtcccttgtcctctcagggttttgatacagtatctgtaaattgaagatattgggctggatcaggtacattttatcttaaggaccaattccaatccattggtagtgggtgcccagtgc

SEQ ID NO: 11 - DNA, 535 bp, Homo sapiens
  wild-type 3′ untranslated region of NOD2/CAD15 (_00STD)
tcactaaccagctcaggaagctcaccagcttgggaagttaatcattatgtctagcttcagtttctcctgcttcagtttaaattgggaaagagagagaaaaaatattcactcattatctgtttcctaaaattgtccttaacatccttcctcttactcctttattacctggtcgggcttcccctcttcaggcgaaatctgtcagtctatctgcattgccttttgatctctacttcagttactacaacttcaaagacaccattgtcctccccaaggtgaggcccatgtagagaaaggatcacttccttgctgaaagagagggtcaaggggcgacccacgtgggccctccctgaaacccaggcccaggcctgagcctggacacctccttccttcctgagaccacagccagcccggtttctctggggccaagagcaaatgctttgcttaagtgctgaaatctcagcccactgaccccttgcagacaggagaggaggggaagcccagggaagctcaacttcccaagtgtcctgagtctctg

SEQ ID NO: 12 - DNA, 496 bp, Homo sapiens
  3′ untranslated region of NOD2/CAD15 _01SS7
aatcattatgtctagcttcagtttctcctgcttcagtttaaattgggaaagagagagaaaaaatattcactcattatctgtttcctaaaattgtccttaacatccttcctcttactcctttattacctggtcgggcttcccctcttcaggcgaaatctgtcagtctatctgcattgccttttgatctctacttcagttactacmacttcaaagacaccattgtcctccccaaggtgaggsccatgtagagaaaggatcacttccttgctgaaagagagggtcaaggggtgacccacgtgggccctccctgaaacccaggcccaggcctgagcctggacacctccttccttcctgagaccacagccagcccggtttctctggggccaagagcaaatgctttgcttaagtgctgaaatctcagcccactgaccccttgcmgacaggagaggaggggaagcccagggaagctcaacttcccaagtgtcctgagtctctg

SEQ ID NO: 13 - DNA, 488 bp, Homo sapiens
  3′ untranslated region of NOD2/CAD15 _02SS7
tgtctagcttcagtttctcctgcttcagtttaaattgggaaagagagagaaaaaatattcactcattatctgtttcctaaaattgtccttaacatccttcctcttactcctttattacctggtcgggcttcccctcttcaggcgaaatctgtcagtctatctgcattgccttttgatctctacttcagttactacaacttcaaagacaccattgtcctccccaaggtgaggcccatgtagagaaaggatcacttccttgctgaaagagagggtcaaggggtgacccacgtgggccctccctgaaacccaggcccaggcctgagcctggacacctccttccttcctgagaccacagccagcccggtttctctggggccaagagcaaatgctttgcttaagtgctgaaatctcagcccactgamcccttgcagacaggagaggaggggaagcccagggaagctcaacttcccaagtgtcctgagtctctg

SEQ ID NO: 14 - DNA, 491 bp, Homo sapiens
  3′ untranslated region of NOD2/CAD15 _03SS6
ttatgtctagcttcagtttctcctgcttcagtttaaattgggaaagagagagaaaaaatattcactcattatctgtttcctaaaattgtccttaacatccttcctcttactcctttattacctggtcgggcttcccctcttcaggcgaaatctgtcagtctatctgcattgccttttgatctctacttcagttactacaacttcaaagacaccattgtcctccccaaggtgaggcccatgtagagaaaggatcacttccttgctgaaagagagggtcaaggggygacccacgtgggccctccctgaaacccaggcccaggcctgagcctggacacctccttccttcctgagaccacagccagcccggtttctctggggccaagagcaaatgctttgcttaagtgctgaaatctcagcccactgaccccttgcagacaggagaggaggggaagcccagggaagctcaacttcccaagtgtcctgagtctctg

SEQ ID NO: 15 - DNA, 491 bp, Homo sapiens; identical to SEQ ID NO: 14
  3′ untranslated region of NOD2/CAD15 _04SS7

SEQ ID NO: 16 - DNA, 491 bp, Homo sapiens; identical to SEQ ID NO: 14
  3′ untranslated region of NOD2/CAD15 _05SS7

SEQ ID NO: 17 - DNA, 491 bp, Homo sapiens
  3′ untranslated region of NOD2/CAD15 _06SS2
ttatgtctagcttcagtttctcctgcttcagtttaaattgggaaagagagagaaaaaatattcactcattatctgtttcctaaaattgtccttaacatccttcctcttactcctttattacctggtcgggcttcccctcttcaggcgaaatctgtcagnctatctgcattgccttttgatctctacttcagttactacaacttcaaagacaccattgtcctccccaaggtgaggcccatgtagagaaaggatcacttccttgctgaaagagagggtcaaggggygacccacgtgggccctccctgaaacccaggcccaggcctgagcctggacacctccttccttcctgagaccacagccagcccggtttctctggggccaagagcaaatgctttgcttaagtgctgaaatctcagcccactgaccccttgcagacaggagaggaggggaagcccagggaagctcaacttcccaagtgtcctgagtctctg

SEQ ID NO: 18 - DNA, 487 bp, Homo sapiens
  3′ untranslated region of NOD2/CAD15 _08SS7
gtctagcttcagtttctcctgcttcagtttaaattgggaaagagagagaaaaaatattcactyattatctgtttcctaaaattgtccttaacatccttcctcttactcctttattacctggtcgggcttcccctcttcaggcgaaatctgtcagtctatctgcattgccttttgatctctacttcagttactacaacttcaaagacaccattgtcctccccaaggtgaggcccatgtagagaaaggatcacttccttgctgaaagagagggtcaaggggygacccacgtgggccctccctgaaacccaggcccaggcctgagcctggacacctccttccttcctgagaccacagccagcccggtttctctggggccaagagcaaatgctttgcttaagtgctgaaatctcagcccactgaccccttgcagacaggagaggaggggaagcccagggaagctcaacttcccaagtgtcctgagtctctg

SEQ ID NO: 19 - DNA, 486 bp, Homo sapiens
  3′ untranslated region of NOD2/CAD15 _12SS7
tctagcttcagtttctcctgcttcagtttaaattgggaaagagagagaaaaaatattcactcattatctgtttcctaaaattgtccttaacatccttcctcttactcctttattacctggtcgggcttcccctcttcaggcgaaatctgtcagtctatctgcattgccttttgatctctacttcagttactacaacttcaaagacaccattgtcctccccaaggtgaggcccatgtagagaaaggatcacttccttgctgaaagagagggtcaaggggcgacccacgtgggccctccctgaaacccaggcccaggcctgagcctggacacctccttccttcctgagaccacagccagcccggtttctctggggccaagagcaaatgctttgcttaagtgctgaaatctcagcccactgaccccttgcagacaggagaggaggggaagcccagggaagctcaacttcccaagtgtcctgagtctctg

SEQ ID NO: 20 - DNA, 484 bp, Homo sapiens
  3′ untranslated region of NOD2/CAD15 _13SS7
tagcttcagtttctcctgcttcagtttaaattgggaaagagagagaaaaaatattcactcattatctgtttcctaaaattgtccttaacatccttcctcttactcctttattacctggtcgggcttcccctcttcaggcgaaatctgtcagtctatctgcattgccttttgatctctacttcagttactacaacttcaaagacaccattgtcctccccaaggtgaggcccatgtagagaaaggatcacttccttgctgaaagagagggtcaaggggcgacccacgtgggccctccctgaaacccaggcccaggcctgagcctggacacctccttccttcctgagaccacagccagcccggtttctctggggccaagagcaaatgctttgcttaagtgctgaaatctcagcccactgaccccttgcagacaggagaggaggggaagcccagggaagctcaacttcccaagtgtcctgagtctctg

SEQ ID NO: 21 - DNA, 485 bp, Homo sapiens
  3′ untranslated region of NOD2/CAD15 _14SS7
ctagcttcagtttctcctgcttcagtttaaattgggaaagagagagaaaaaatattcactyattatctgtttcctaaaattgtccttaacatccttcctcttactcctttattacctggtcgggcttcccctcttcaggcgaaatctgtcagtctatctgcattgccttttgatctctacttcagttactacaacttcaaagacaccattgtcctccccaaggtgaggcccatgtagagaaaggatcacttccttgctgaaagagagggtcaaggggcgacccacgtgggccctccctgaaacccaggcccaggcctgagcctggacacctccttccttcctgagaccacagccagcccggtttctctggggccaagagcaaatgctttgcttaagtgctgaaatctcagcccactgaccccttgcagacaggagaggaggggaagcccagggaagctcaacttcccaagtgtcctgagtctctg

SEQ ID NO: 22 - DNA, 488 bp, Homo sapiens
  3′ untranslated region of NOD2/CAD15 _19SS7
tgtctagcttcagtttctcctgcttcagtttaaattgggaaagagagagaaaaaatattcacttattatctgtttcctaaaattgtccttaacatccttcctcttactcctttattacctggtcgggcttcccctcttcaggcgaaatctgtcagtctatctgcattgccttttgatctctacttcagttactacaacttcaaagacaccattgtcctccccaaggtgaggcccatgtagagaaaggatcacttccttgctgaaagagagggtcaaggggcgacccacgtgggccctccctgaaacccaggcccaggcctgagcctggacacctccttccttcctgagaccacagccagcccggtttctctggggccaagagcaaatgctttgcttaagtgctgaaatctcagcccactgaccccttgcagacaggagaggaggggaagcccagggaagctcaacttcccaagtgtcctgagtctctg

SEQ ID NO: 23 - DNA, 488 bp, Homo sapiens; identical to SEQ ID NO: 22
  3′ untranslated region of NOD2/CAD15 _22SS7

SEQ ID NO: 24 - DNA, 533 bp, Homo sapiens
  wild-type 3′ untranslated region of NOD2/CAD15 (_00STD)
ttcgtctcagtttgtttgtgagcaggctgtgagtttgggccccagaggctgggtgacatgtgttggcagcctcttcaaaatgagccctgtcctgcctaaggctgaacttgttttctgggaacaccataggtcacctttattctggcagaggagggagcatcagtgccctccaggatagacttttcccaagcctacttttgccattgacttcttcccaagattcaatcccaggatgtacaaggacagcccctcctccatagtatgggactggcctctgctgatcctcccaggcttccgtgtgggtcagtggggcccatggatgtgcttgttaactgagtgccttttggtggagaggcccggcctctcacaaaagaccccttaccactgctctgatgaagaggagtacacagaacacataattcaggaagcagctttccccatgtctcgactcatccatccaggccattccccgtctctggttcctcccctcctcctggactcctgcacacgctccttcctctgaggctgaaatt

SEQ ID NO: 25 - DNA, 497 bp, Homo sapiens
  3′ untranslated region of NOD2/CAD15 _01SS6
gggccccagaggctgggtgacatgtgttggcagcctcttcaaaatgagccctgtcctgcctaaggctgaacttgttttctgggaacaccataggtcacctttattctggcagaggagggagcatcagtgccctccaggatagacttttcccaagcctacttttgccattgacttcttcccaagattcaatcccaggatgtacaaggacagcccctcctccatagtatgggactggcctctgctgatcctcccaggcttccgtgtgggtcagtggggcccatggatgtgcttgttaactgagtgccttttggtggagaggcccggcctctcacaaaagaccccttaccactgctctgatgaagaggagtacacagaacacataattcaggaagcagctttccccatgtctcgactcatccatccaggccattccccgtctctggttcctcccctcctcctggactcctgcacacgctccttcctctgaggctgaaatt

SEQ ID NO: 26 - DNA, 497 bp, Homo sapiens
  3′ untranslated region of NOD2/CAD15 _02SS6
gggccccagaggctgggtgacatgtgttggaagcctcttcaaaatgagccctgtcctgcctaaggctgaacttgttttctgggaacaccataggtcacctttattctggcagaggagggagcatcagtgccctccaggatagacttttcccaagcctacttttgccattgacttcttcccaagattcaatcccaggatgtacaaggacagcccctcctccatagtatgggactggcctctgctgatcctcccaggcttccgtgtgggtcagtggggcccatggatgtgcttgttaactgagtgccttttggtggagaggcccggcctctcacaaaagaccccttaccactgctctgatgaagaggagtacacagaacacataattcaggaagcagctttccccatgtctcgactcatccatccaggccattccccgtctctggttcctcccctcctcctggactcctgcacacgctccttcctctgaggctgaaatt

SEQ ID NO: 27 - DNA, 497 bp, Homo sapiens
  3′ untranslated region of NOD2/CAD15 _03SS6
gggccccagaggctgggtgacatgtgttggcagcctcttcaaaatgagccctgtcctgcctaaggctgaacttgttttctgggaacaccataggtcacctttattctggcagaggagggagcatcagtgccctccaggatagacttttcccaagcctacttttgccattgacttcttcccaagattcaatcccaggatgtacaaggacagcccctcctccatagtatgggactggcctctgctgatcctcccaggcttccgtgtgggtcagtggggcccatggatgtgcttgttaactgagtgccttttggtggagaggcccggcctctcacaaaagaccccttaccactgctctgatgaagaggagtacacagaacacataattcaggaagcagctttccccatgtctcgactcatccatccaggccattccccgtctctggttcctcccctcctcctggactcctgcacacgctccttcctctgagggtgaaatt

SEQ ID NO: 28 - DNA, 483 bp, Homo sapiens
  3′ untranslated region of NOD2/CAD15 _04SS6
gggtgacatgtgttggcagcctcttcaaaatgagccctgtcctgcctaaggctgaacttgttttctgggaacaccataggtcacctttattctggcagaggagggagcatcagtgccctccaggatagacttttcccaagcctacttttgccattgacttcttcccaagattcaatcccaggatgtacaaggacagcccctcctccatagtatgggactggcctctgctgatcctcccaggcttccgtgtgggtcagtggggcccatggatgtgcttgttaactgagtgccttttggtggagaggcccggcctctcacaaaagaccccttaccactgctctgatgaagaggagtacacagaacacmtaattcaggaagcagctttccccatgtctcgactcatccatccaggccattccccgtctctggttcctcccctcctcctggactcctgcacacgctccttcctctgaggctgaaatt

SEQ ID NO: 29 - DNA, 500 bp, Homo sapiens
  3′ untranslated region of NOD2/CAD15 _05SS2
tttgggccccagaggctgggtgacatgtgttggcagcctcttcaaaatgagccctgtcctgcctaaggctgaacttgttttctgggaacaccataggtcacctttattctggcagaggagggagcatcagtgccctccaggatagacttttcccaagcctacttttgccattgacttcttcccaagattcaatcccaggatgtacaaggacagcccctcctccatagtatgggactggcctctgctgatcctcccaggcttccgtgtgggtcagtggggcccatggatgtgcttgttaactgagtgccttttggtggagaggcccggcctctcacaaaagaccccttmccactgctctgatgaagaggagtacacagaacacataattcaggaagcagctttccccatgtctcgactcatccatccaggccattccccgtctctggttcctcccctcctcctggactcctgcacacgctccttcctctgaggctgaaatt

SEQ ID NO: 30 - DNA, 499 bp, Homo sapiens
  3′ untranslated region of NOD2/CAD15 _06SS6
ttgggccccagaggctgggtgacatgtgttggcagcctcttcaaaatgagccctgtcctgcctaaggctgaacttgttttctgggaacaccataggtcacctttattctggcagaggagggagcatcagtgccctccaggatagacttttcccaagcctacttttgccattgacttcttcccaagattcaatcccaggatgtacaaggacagcccctcctccatagtatgggactggcctctgctgatcctcccaggcttccgtgtgggtcagtggggcccatggatgtgcttgttaactgagtgccttttggtggagaggcccggcctctcacaaaagaccccttmccactgctctgatgaagaggagtacacagaacacataattcaggaagcagctttccccatgtctcgactcatccatccaggccattccccgtctctggttcctcccctcctcctggactcctgcacacgctccttcctctgaggctgaaatt

SEQ ID NO: 31 - DNA, 498 bp, Homo sapiens
  3′ untranslated region of NOD2/CAD15 _08SS6
tgggccccagaggctgggtgacatgtgttggcagcctcttcaaaatgagccctgtcctgcctaaggctgaacttgttttctgggaacaccataggtcacctttattctggcagaggagggagcatcagtgccctccaggatagacttttcccaagcctacttttgccattgacttcttcccaagattcaatcccaggatgtacaaggacagcccctcctccatagtatgggactgggctctgctgatcctcccaggcttccgtgtgggtcagtggggcccatggatgtgcttgttaactgagtgccttttggtggagaggcccggcctctcacaaaagaccccttmccactgctctgatgaagaggagtacacagaacacataattcaggaagcagctttccccatgtctcgactcatccatccaggccattccccgtctctggttcctcccctcctcctggactcctgcacacgctccttcctctgaggctgaaatt

SEQ ID NO: 32 - DNA, 498 bp, Homo sapiens
  3′ untranslated region of NOD2/CAD15 _12SS6
tgggccccagaggctgggtgacatgtgttggcagcctcttcaaaatgagccctgtcctgcctaaggctgaacttgttttctgggaacaccataggtcacctttattctggcagaggagggagcatcagtgccctccaggatagacttttcccaagcctacttttgccattgacttcttcccaagattcaatcccaggatgtacaaggacagcccctcctccatagtatgggactggcctctgctgatcctcccaggcttccgtgtgggtcagtggggcccatggatgtgcttgttaactgagtgccttttggtggagaggcccggcctctcacaaaagaccccttmccactgctctgatgaagaggagtacacagaacacataattcaggaagcagctttccccatgtctcgactcatccatccaggccattccccgtctctggttcctcccctcctcctggactcctgcacacgctccttcctctgaggctgaaatt

SEQ ID NO: 33 - DNA, 498 bp, Homo sapiens
  3′ untranslated region of NOD2/CAD15 _13SS6
tgggccccagaggctgggtgacatgtgttggcagcctcttcaaaatgagccctgtcctgcctaaggctgaacttgttttctgggaacaccataggtcacctttattctggcagaggagggagcatcagtgccctccaggatagacttttcccaagcctacttttgccattgacttcttcccaagattcaatcccaggatgtacaaggacagcccctcctccatagtatgggactggcctctgctgatcctcccaggcttccgtgtgggtcagtggggcccatggatgtgcttgttaactgagtgccttttggtggagaggcccggcctctcacaaaagaccccttaccactgctctgatgaagaggagtacacagaacacataattcaggaagcagctttccccatgtctcgactcatccatccaggccattccccgtctctggttcctcccctcctcctggactcctgcacacgctccttcctctgaggctgaaatt

SEQ ID NO: 34 - DNA, 503 bp, Homo sapiens
  3′ untranslated region of NOD2/CAD15 _14SS6
gagtttgggccccagaggctgggtgacatgtgttggcagcctcttcaaaatgagccctgtcctgcctaaggctgaacttgttttctgggaacaccataggtcacctttattctggcagaggagggagcatcagtgccctccaggatagacttttcccaagcctacttttgccattgacttcttcccaagattcaatcccaggatgtacaaggacagcccctcctccatagtatgggactggcctctgctgatcctcccaggcttccgtgtgggtcagtggggcccatggatgtgcttgttaactgagtgccttttggtggagaggcccggcctctcacaaaagaccccttcccactgctctgatgaagaggagtacacagaacacataattcaggaagcagctttccccatgtctcgactcatccatccaggccattccccgtctctggttcctcccctcctcctggactcctgcacacgctccttcctctgaggctgaaatt

SEQ ID NO: 35 - DNA, 453 bp, Homo sapiens
  3′ untranslated region of NOD2/CAD15 _19SS6
tgagccctgtcctgcctaaggctgaacttgttttctgggaacaccataggtcacctttattctggcagaggagggagcatcagtgccctccaggatagacttttcccaagcctacttttgccattgacttcttcccaagattcaatcccaggatgtacaaggacagcccctcctccatagtatgggactggcctctgctgatcctcccaggcttccgtgtgggtcagtggggcccatggatgtgcttgttaactgagtgccttttggtggagaggcccsgcctctcacaaaagaccccttcccactgctctgatgaagaggagtacacagaacacataattcaggaagcagctttccccatgtctcgactcatccatccaggccattccccgtctctggttcctcccctcctcctggactcctgcacacgctccttcctctgaggctgaaatt

SEQ ID NO: 36 - DNA, 500 bp, Homo sapiens
  3′ untranslated region of NOD2/CAD15 _22SS6
tttgggccccagaggctgggtgacatgtgttggcagcctcttcaaaatgagccctgtcctgcctaaggctgaacttgttttctgggaacaccataggtcacctttattctggcagaggagggagcatcagtgccctccaggatagacttttcccaagcctacttttgccattgacttcttcccaagattcaatcccaggatgtacaaggacagcccctcctccatagtatgggactggcctctgctgatcctcccaggcttccgtgtgggtcagtggggcccatggatgtgcttgttaactgagtgccttttggtggagaggcccggcctctcacaaaagaccccttmccactgctctgatgaagaggagtacacagaacacataattcaggaagcagctttccccatgtctcgactcatccatccaggccattccccgtctctggttcctcccctcctcctggactcctgcacacgctccttcctctgagggtgaaatt

SEQ ID NO: 37 - DNA, 13 bp, Artificial Sequence
  synthetic Taqman probe for NOD2/CARD15 SNP5 wild type allele (“1”)
catggctggaccc

SEQ ID NO: 38 - DNA, 13 bp, Artificial Sequence
  synthetic Taqman probe for NOD2/CARD15 SNP5 variant allele (“2”)
catggctggatcc

SEQ ID NO: 39 - DNA, 13 bp, Artificial Sequence
  synthetic Taqman probe for NOD2/CARD15 SNP8 wild type allele (“1”)
tgctccggcgcca

SEQ ID NO: 40 - DNA, 14 bp, Artificial Sequence
  synthetic Taqman probe for NOD2/CARD15 SNP8 variant allele (“2”)
ctgctctggcgcca

SEQ ID NO: 41 - DNA, 16 bp, Artificial Sequence
  synthetic Taqman probe for NOD2/CARD15 SNP12 wild type allele (“1”)
ctctgttgccccagaa

SEQ ID NO: 42 - DNA, 15 bp, Artificial Sequence
  synthetic Taqman probe for NOD2/CARD15 SNP12 variant allele (“2”)
ctctgttgcgccaga

SEQ ID NO: 43 - DNA, 15 bp, Artificial Sequence
  synthetic Taqman probe for NOD2/CARD15 SNP13 wild type allele (“1”)
ctttcaagggcctgc

SEQ ID NO: 44 - DNA, 15 bp, Artificial Sequence
  synthetic Taqman probe for NOD2/CARD15 SNP13 variant allele (“2”)
cctttcaaggggcct

SEQ ID NO: 45 - DNA, 16 bp, Artificial Sequence
  synthetic Taqman probe for NOD2/CARD15 JW1 wild type allele
aagactcgagtgtcct

SEQ ID NO: 46 - DNA, 16 bp, Artificial Sequence
  synthetic Taqman probe for NOD2/CARD15 JW1 variant allele
agactcaagtgtcctc

SEQ ID NO: 47 - DNA, 24 bp, Artificial Sequence
  synthetic NOD2/CARD15 sequencing forward primer for exon 1 (Ex1)
tctcctccccagatgtttaagatg

SEQ ID NO: 48 - DNA, 20 bp, Artificial Sequence
  synthetic NOD2/CARD15 sequencing reverse primer for exon 1 (Ex1)
ccagccaaggatgccacagc

SEQ ID NO: 49 - DNA, 20 bp, Artificial Sequence
  synthetic NOD2/CARD15 sequencing forward primer for exon 2 (Ex2)
tgccttctctgggtctcaat

SEQ ID NO: 50 - DNA, 20 bp, Artificial Sequence
  synthetic NOD2/CARD15 sequencing reverse primer for exon 2 (Ex2)
atggaccaagttaccccaca

SEQ ID NO: 51 - DNA, 19 bp, Artificial Sequence
  synthetic NOD2/CARD15 sequencing forward primer for exon 3 (Ex3)
gactgcccttccctttctg

SEQ ID NO: 52 - DNA, 20 bp, Artificial Sequence
  synthetic NOD2/CARD15 sequencing reverse primer for exon 3 (Ex3)
acattgctccatcagccttc

SEQ ID NO: 53 - DNA, 20 bp, Artificial Sequence
  synthetic NOD2/CARD15 sequencing forward primer for exon 4 (Ex4D)
cagagccccttcccgtcatc

SEQ ID NO: 54 - DNA, 24 bp, Artificial Sequence
  synthetic NOD2/CARD15 sequencing reverse primer for exon 4 (Ex4D)
agcacagtgtccgcatcgtcattg

SEQ ID NO: 55 - DNA, 20 bp, Artificial Sequence
  synthetic NOD2/CARD15 sequencing forward primer for exon 4 (Ex4C)
ctggaggagctcttcagcac

SEQ ID NO: 56 - DNA, 20 bp, Artificial Sequence
  synthetic NOD2/CARD15 sequencing reverse primer for exon 4 (Ex4C)
aacagttcctggtggcattt

SEQ ID NO: 57 - DNA, 20 bp, Artificial Sequence
  synthetic NOD2/CARD15 sequencing forward primer for exon 4 (Ex4B)
cctgctccaagagacctcag

SEQ ID NO: 58 - DNA, 20 bp, Artificial Sequence
  synthetic NOD2/CARD15 sequencing reverse primer for exon 4 (Ex4B)
tcagatgtctggcactcagc

SEQ ID NO: 59 - DNA, 20 bp, Artificial Sequence
  synthetic NOD2/CARD15 sequencing forward primer for exon 4 (Ex4A)
agatcacagcagccttcctg

SEQ ID NO: 60 - DNA, 20 bp, Artificial Sequence
  synthetic NOD2/CARD15 sequencing reverse primer for exon 4 (Ex4A)
atctgggcagtgttgcaaag

SEQ ID NO: 61 - DNA, 20 bp, Artificial Sequence
  synthetic NOD2/CARD15 sequencing forward primer for exons 5 and 6 (Ex5&6)
ttttgggggatttgtagatt

SEQ ID NO: 62 - DNA, 24 bp, Artificial Sequence
  synthetic NOD2/CARD15 sequencing reverse primer for exons 5 and 6 (Ex5&6)
ctggggagatcacagcattagaga

SEQ ID NO: 63 - DNA, 19 bp, Artificial Sequence
  synthetic NOD2/CARD15 sequencing forward primer for exon 7 (Ex7)
actctctccctggcttgtc

SEQ ID NO: 64 - DNA, 18 bp, Artificial Sequence
  synthetic NOD2/CARD15 sequencing reverse primer for exon 7 (Ex7)
cgtcccgctgcccctttc

SEQ ID NO: 65 - DNA, 20 bp, Artificial Sequence
  synthetic NOD2/CARD15 sequencing forward primer for exon 8 (Ex8)
gaggccactctgggattgag

SEQ ID NO: 66 - DNA, 21 bp, Artificial Sequence
  synthetic NOD2/CARD15 sequencing reverse primer for exon 8 (Ex8)
cctgatccagcccaatatctt

SEQ ID NO: 67 - DNA, 21 bp, Artificial Sequence
  synthetic NOD2/CARD15 sequencing forward primer for exon 9 (Ex9)
tgccaggcactatattaaggt

SEQ ID NO: 68 - DNA, 19 bp, Artificial Sequence
  synthetic NOD2/CARD15 sequencing reverse primer for exon 9 (Ex9)
gggctggatcaggtacatt

SEQ ID NO: 69 - DNA, 22 bp, Artificial Sequence
  synthetic NOD2/CARD15 sequencing forward primer for exon 10 (Ex10)
ctttattggttaccttcacttc

SEQ ID NO: 70 - DNA, 18 bp, Artificial Sequence
  synthetic NOD2/CARD15 sequencing reverse primer for exon 10 (Ex10)
gctgcaatggagagtggg

SEQ ID NO: 71 - DNA, 18 bp, Artificial Sequence
  synthetic NOD2/CARD15 sequencing forward primer for exon 11 (Ex11)
gatggcacgggtactctt

SEQ ID NO: 72 - DNA, 21 bp, Artificial Sequence
  synthetic NOD2/CARD15 sequencing reverse primer for exon 11 (Ex11)
actgaggttcggagagctaaa

SEQ ID NO: 73 - DNA, 21 bp, Artificial Sequence
  synthetic NOD2/CARD15 sequencing forward primer for exon 12 (Ex12)
gagggcaccagggtttgctca

SEQ ID NO: 74 - DNA, 24 bp, Artificial Sequence
  synthetic NOD2/CARD15 sequencing reverse primer for exon 12 (Ex12)
gatcagcagaggccagtcccatac

SEQ ID NO: 75 - DNA, 18 bp, Artificial Sequence
  synthetic NOD2/CARD15 sequencing forward nested primer for exon 9 (Ex9 nested)
ccccagagcagagaatcc

SEQ ID NO: 76 - DNA, 20 bp, Artificial Sequence
  synthetic NOD2/CARD15 sequencing reverse nested primer for exon 9 (Ex9 nested)
ctttccctgctctgacatac

SEQ ID NO: 77 - DNA, 497 bp, Homo sapiens
  wild-type 3′ untranslated region of NOD2/CAD15 (00STD)
tcactaggcttctggttgatgcctgtgaactgaactctgacaacagacttctgaaatagacccacaagaggcagttccatttcatttgtgccagaatgctttaggatgtacagttatggattgaaagtttacaggaaaaaaaattaggccgttccttcaaagcaaatgtcttcctggattattcaaaatgatgtatgttgaagcctttgtaaattgtcagatgctgtgcaaatgttattattttaaacattatgatgtgtgaaaactggttaatatttataggtcactttgttttactgtcttaagtttatactcttatagacaacatggccgtgaactttatgctgtaaataatcagaggggaataaactgttgagtcaaaacagccatcttccttgtgaccaaacatttaaaaatattctggctgggcacagtggctcacgcctgtaatcccagcactttgggaggccgaggtgggcagatcacctgaggttggg

SEQ ID NO: 78 - DNA, 460 bp, Homo sapiens
  3′ untranslated region of NOD2/CAD15 (_01QSS6)
tgacaacagacttctgaaatagacccacaagaggcagttccatttcatttgtgccagaatgctttaggatgtacagttatggattgaaagtttacaggaaaaaaaattaggccgttccttcaaagcaaatgtcttcctggattattcaaaatgatgtatgttgaagcctttgtaaattgtcagatgctgtgcaaatgttattattttaaacattatgatgtgtgaaaactggttaatatttataggtcactttgttttactgtcttaagtttatactcttatagacaacatggccgtgaactttatgctgtaaataatcagaggggaataaactgttgagtcaaaacagccatcttccttgtgaccaaacatttaaaaatattctggctgggcacagtggctcacgcctgtaatcccagcactttgggaggccgaggtgggcagatcacctgaggttggg

SEQ ID NO: 79 - DNA, 462 bp, Homo sapiens
  3′ untranslated region of NOD2/CAD15 (_02SS6)
tctgacaacagacttctgaaatagacccacaagaggcagttccatttcatttgtgccagaatgctttaggatgtacagttatggattgaaagtttacaggaaaaaaaattaggccgttccttcaaagcaaatgtcttcctggattattcaaaatgatgtatgttgaagcctttgtaaattgtcagatgctgtgcaaatgttattattttaaacattatgatgtgtgaaaactggttaatatttataggtcactttgttttactgtcttaagtttatactcttatagacaacatggccgtgaactttatgctgtaaataatcagaggggaataaactgttgagtcaaaacagccatcttccttgtgaccaaacatttaaaaatattctggctgggcacagtggctcacgcctgtaatcccagcactttgggaggccgaggtgggcagatcacctgaggttggg

SEQ ID NO: 80 - DNA, 459 bp, Homo sapiens
  3′ untranslated region of NOD2/CAD15 (_03SS6)
gacaacagacttctgaaatagacccacaagaggcagttccatttcatttgtgccagaatgctttaggatgtacagttatggattgaaagtttacaggaaaaaaaattaggccgttccttcaaagcaaatgtcttcctggattattcaaaatgatgtatgttgaagcctttgtaaattgtcagatgctgtgcaaatgttattattttaaacattatgatgtgtgaaaactggttaatatttataggtcactttgttttactgtcttaagtttatactcttatagacaacatggccgtgaactttatgctgtaaataatcagaggggaataaactgttgagtcaaaacagccatcttccttgtgaccaaacatttaaaaatattctggctgggcacagtggctcacgcctgtaatcccagcactttgggaggccgaggtgggcagatcacctgaggttggg

SEQ ID NO: 81 - DNA, 467 bp, Homo sapiens
  3′ untranslated region of NOD2/CAD15 (_04SS6)
tgaactctgacaacagacttctgaaatagacccacaagaggcagttccatttcatttgtgccagaatgctttaggatgtacagttatggattgaaagtttacaggaaaaaaaattaggccgttccttcaaagcaaatgtcttcctggattattcaaaatgatgtatgttgaagcctttgtaaattgtcagatgctgtgcaaatgttattattttaaacattatgatgtgtgaaaactggttaatatttataggtcactttgttttactgtcttaagtttatactcttatagacaacatggccgtgaactttatgctgtaaataatcagaggggaataaactgttgagtcaaaacagccatcttccttgtgaccaaacatttaaaaatattctggctgggcacagtggctcacgcctgtaatcccagcactttgggaggccgaggtgggcagatcacctgaggttggg

SEQ ID NO: 82 - DNA, 467 bp, Homo sapiens
  3′ untranslated region of NOD2/CAD15 (_05SS2)
tgaactctgacaacagacttctgaaatagacccacaagaggcagttccatttcatttgtgccagaatgctttaggatgtacagttatggattgaaagtttacaggaaaaaaaattaggccgttccttcaaagcaaatgtcttcctggattattcaaaatgatgtatgttgaagcctttgtaaattgtcagatgctgtgcaaatgttattattttaaacattatgatgtgtgaaaactggttaatatttatagrtcactttgttttactgtcttaagtttatactcttatagacaacatggccgtgaactttatgctgtaaataatcagaggggaataaactgttgagtcaaaacagccatcttccttgtgaccaaacatttaaaaatattctggctgggcacagtggctcacgcctgtaatcccagcactttgggaggccgaggtgggcagatcacctgaggttggg

SEQ ID NO: 83 - DNA, 466 bp, Homo sapiens
  3′ untranslated region of NOD2/CAD15 (_06SS6)
gaactatgacaacagacttctgaaatagacccacaagaggcagttccatttcatttgtgccagaatgctttaggatgtacagttatggattgaaagtttacaggaaaaaaaattaggccgttccttcaaagcaaatgtcttcctggattattcaaaatgatgtatgttgaagcctttgtaaattgtcagatgctgtgcaaatgttattattttaaacattatgatgtgtgaaaactggttaatatttatagrtcactttgttttactgtcttaagtttatactcttatagacaacatggccgtgaactttatgctgtaaataatcagaggggaataaactgttgagtcaaaacagccatcttccttgtgaccaaacatttaaaaatattctggctgggcacagtggctcacgcctgtaatcccagcactttgggaggccgaggtgggcagatcacctgaggttggg

SEQ ID NO: 84 - DNA, 466 bp, Homo sapiens
  3′ untranslated region of NOD2/CAD15 (_08SS6)
gaactctgacaacagacttctgaaatagacccacaagaggcagttccatttcatttgtgccagaatgctttaggatgtacagttatggattgaaagtttacaggaaaaaaaattaggccgttccttcaaagcaaatgtcttcctggattattcaaaatgatgtatgttgaagcctttgtaaattgtcagatgctgtgcaaatgttattattttaaacattatgatgtgtgaaaactggttaatatttatagrtcactttgttttactgtcttaagtttatactcttatagacaacatggccgtgaactttatgctgtaaataatcagaggggaataaactgttgagtcaaaacagccatcttccttgtgaccaaacatttaaaaatattctggctgggcacagtggctcacgcctgtaatcccagcactttgggaggccgaggtgggcagatcacctgaggttggg

SEQ ID NO: 85 - DNA, 460 bp, Homo sapiens
  3′ untranslated region of NOD2/CAD15 (_12SS6)
tgacaacagacttctgaaatagacccacaagaggcagttccatttcatttgtgccagaatgctttaggatgtacagttatggattgaaagtttacaggaaaaaaaattaggccgttccttcaaagcaaatgtcttcctggattattcaaaatgatgtatgttgaagcctttgtaaattgtcagatgctgtgcaaatgttattattttaaacattatgatgtgtgaaaactggttaatatttatagrtcactttgttttactgtcttaagtttatactcttatagacaacatggccgtgaactttatgctgtaaataatcagaggggaataaactgttgagtcaaaacagccatcttccttgtgaccaaacatttaaaaatattctggctgggcacagtggctcacgcctgtaatcccagcactttgggaggccgaggtgggcagatcacctgaggttggg

SEQ ID NO: 86 - DNA, 467 bp, Homo sapiens; identical to SEQ ID NO: 81
  3′ untranslated region of NOD2/CAD15 (_13SS6)

SEQ ID NO: 87 - DNA, 460 bp, Homo sapiens
  3′ untranslated region of NOD2/CAD15 (_14SS6)
tgacaacagacttctgaaatagacccacaagaggcagttccatttcatttgtgccagaatgctttaggatgtacagttatggattgaaagtttacaggaaaaaaaattaggccgttccttcaaagcaaatgtcttcctggattattcaaaatgatgtatgttgaagcctttgtaaattgtcagatgctgtgcaaatgttattattttaaacattatgatgtgtgaaaactggttaatatttatagatcactttgttttactgtcttaagtttatactcttatagacaacatggccgtgaactttatgctgtaaataatcagaggggaataaactgttgagtcaaaacagccatcttccttgtgaccaaacatttaaaaatattctggctgggcacagtggctcacgcctgtaatcccagcactttgggaggccgaggtgggcagatcacctgaggttggg

SEQ ID NO: 88 - DNA, 462 bp, Homo sapiens
  3′ untranslated region of NOD2/CAD15 (_19SS6)
tctgacaacagacttctgaaatagacccacaagaggcagttccatttcatttgtgccagaatgctttaggatgtacagttatggattgaaagtttacaggaaaaaaaattaggccgttccttcaaagcaaatgtcttcctggattattcaaaatgatgtatgttgaagcctttgtaaattgtcagatgctgtgcaaatgttattattttaaacattatgatgtgtgaaaactggttaatatttatagatcactttgttttactgtcttaagtttatactcttatagacaacatggccgtgaactttatgctgtaaataatcagaggggaataaactgttgagtcaaaacagccatcttccttgtgaccaaacatttaaaaatattctggctgggcacagtggctcacgcctgtaatcccagcactttgggaggccgaggtgggcagatcacctgaggttggg

SEQ ID NO: 89 - DNA, 463 bp, Homo sapiens
  3′ untranslated region of NOD2/CAD15 (_22SS6)
ctctgacaacagacttctgaaatagacccacaagaggcagttccatttcatttgtgccagaatgctttaggatgtacagttatggattgaaagtttacaggaaaaaaaattaggccgttccttcaaagcaaatgtcttcctggattattcaaaatgatgtatgttgaagcctttgtaaattgtcagatgctgtgcaaatgttattattttaaacattatgatgtgtgaaaactggttaatatttatagrtcactttgttttactgtcttaagtttatactcttatagacaacatggccgtgaactttatgctgtaaataatcagaggggaataaactgttgagtcaaaacagccatcttccttgtgaccaaacatttaaaaatattctggctgggcacagtggctcacgcctgtaatcccagcactttgggaggccgaggtgggcagatcacctgaggttggg